SEQUENCE LISTING (12 sequences)

SEQ ID NO: 1 - protein, 155 aa, Homo sapiens [sequence omitted]

SEQ ID NO: 2 - DNA, 468 bp, Homo sapiens
  source 1..468: mol_type = genomic DNA
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 3 - protein, 476 aa, Homo sapiens [sequence omitted]

SEQ ID NO: 4 - DNA, 1431 bp, Homo sapiens
  source 1..1431: mol_type = genomic DNA
atggactggacctggaggttcctctttgtggtggcagcagctacaggtgtccagtcccagatgcaggtggtgcagtctggggctgaagtaaagaagcctgggtcctcggtgacggtctcctgcaaggcatctggaggcaccttcagcaactatgctatcagctgggtgcgacaggcccctggacaagggcttgagtggatgggagggatcatccctctttttggtacaccaacctactcacagaacttccagggcagagtcacgattaccgcggacaaatccaccagcacagcccacatggagctgatcagcctgagatctgaggacacggccgtgtattactgtgcgacagatcgctacaggcaggcaaattttgaccgggcccgggttggctggttcgacccctggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 235 aa, Homo sapiens [sequence omitted]

SEQ ID NO: 6 - DNA, 708 bp, Homo sapiens
  source 1..708: mol_type = genomic DNA
atggcctgggctctgctgctcctcaccctcctcactcaggacacagggtcctgggcccagtctgccctgactcagcctgcctccgtgtctgggtctcctggacagtcgatcaccatctcctgcactggaaccaacaatgatgttgggagttataaccttgtctcctggtaccagcagcacccaggcaaagcccccaaaatcatgatttatgaggtcagtaagcggccctcaggggtttctaatcgcttctctggctccaagtctggcaacacggcctccctgacaatctctgggctccaggctgaggacgaggctgattattactgctgctcatatgcaggtagttacactgtggttttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 7 - protein, 10 aa, synthetic construct
  VARIANT 1..10: note = This sequence may encompass 2-10 repeating units
HHHHHHHHHH

SEQ ID NO: 8 - protein, 730 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHOURHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 9 - DNA, 2820 bp, Homo sapiens
  source 1..2820: mol_type = genomic DNA
gggagttcagacctagatctttccagttaatcacacaacaaacttagctcatcgcaataaaaagcagctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataaagatttcatgggaaaaaaaatcaattaatctgtctaagctgctttctgatgttggtttcttaataatgagtaaaccacaaattaaatgttattttaacctcaccaaaacaatttataccttgtgtccctaaattgtagccctatattaaattatattacatttcaaaaaaaaaaaaaaaa

SEQ ID NO: 10 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 11 - DNA, 1239 bp, Homo sapiens
  source 1..1239: mol_type = genomic DNA
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 12 - protein, 6 aa, synthetic construct
HHHHHH

(19) United States
(12) Patent Application Publication
(10) Pub. No.: US 2024/0228566 A1
(43) Pub. Date: Jul. 11, 2024

(54) COMPOSITIONS AND METHODS FOR VASCULAR PROTECTION AFTER MYOCARDIAL ISCHEMIA

(71) Applicant: The University of Vermont and State Agriculture College, Burlington, VT (US)

(72) Inventors: Jeffrey L. SPEES, Burlington, VT (US); Benjamin LIEBMAN, Burlington, VT (US)

(73) Assignee: The University of Vermont and State Agriculture College, Burlington, VT (US)

(21) Appl. No.: 18/431,661

(22) Filed: Feb. 2, 2024

Related U.S. Application Data
(63) Continuation of application No. PCT/US2022/074416, filed on Aug. 2, 2022.
(60) Provisional application No. 63/229,138, filed on Aug. 4, 2021.

(51) Int. Cl. C07K 14/475; A61P 9/10; C07K 14/50; C07K 16/22; A61K 38/00

(52) U.S. Cl. CPC C07K 14/4753; A61P 9/10; C07K 14/503; C07K 16/22; A61K 38/00

(57) ABSTRACT

The invention features methods and compositions for treating a reperfusion injury, hypofusion, ischemic injury, and/or low/no-reflow. In embodiments, the compositions contain complexes containing a basic fibroblast growth factor (FGF2) and an immunoglobulin G (IgG) polypeptide, or fragments thereof. In embodiments, the complexes further contain a hepatocyte growth factor (HGF) polypeptide, a vascular endothelial growth factor (VEGF) polypeptide, or fragments thereof.

### CROSS-REFERENCE TO RELATED APPLICATIONS

This application is a continuation under 35 U.S.C. § 111(a) of PCT International Patent Application No. PCT/US2022/074416, filed Aug. 2, 2022, designating the United States and published in English, which claims priority to and the benefit of U.S. Provisional Application No. 63/229,138, filed Aug. 4, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. HL132264 awarded by the National Institutes of Health. The government has certain rights in the invention.

### SEQUENCE LISTING

The present application contains a Sequence Listing which has been submitted electronically in XML format. The entire contents of the electronic XML Sequence Listing. (Date of creation: Sep. 15, 2022; Size: 20,450 bytes; Name: 167914-011601PCT_SL.xml), is incorporated by reference herein.

### BACKGROUND OF THE INVENTION

Coronary heart disease leading to acute myocardial infarction (MI: heart attack) is a principal cause of mortality worldwide. Cornerstone treatments for MI are designed to restore blood flow (i.e. “reperfuse”) blocked coronary arteries. Percutaneous coronary intervention (PCI) involving angioplasty and stent placement is a standard of care treatment to restore blood circulation in the heart after myocardial infarction (MI, heart attack) caused by a thrombus (vascular blood clot). There are about 1 million PCI procedures performed annually in the United States and over 3 million worldwide. Thrombolytics, which enzymatically break down blood clots, are also used to restore blood circulation after MI. Despite reduced times to intervention, and successful stent placement, 30-50% of primary PCI patients exhibit low- or “no-reflow”, a phenomenon linked to poor outcomes, increased probability of heart failure, and death. Low/no-reflow occurs when macroscopic vessels are opened by stenting or thrombolysis, but distal myocardial perfusion remains compromised. The distal myocardial perfusion can be compromised by capillary plugging and microemboli, vascular leak, edema, swelling of the capillary bed, vasospasm, and/or necrotic breakdown/rupture of the microvasculature (alternatively referred to as “vascular rhexis”). All patients undergoing PCI have reperfusion injury that negatively affects their prognosis.

Vascular damage and dysfunction linked to reperfusion injury and no-reflow result in loss of oxygen and nutrients to cardiac myocytes and progressive myocardial necrosis (i.e. death of cardiac tissue). For MI patients, finding new strategies to deal with reperfusion injury and low/no-reflow are a high priority because half of final infarct size is attributed to cardiac tissue damage that occurs post-PCI. Unfortunately, all recent phase III trials to treat low/no-reflow have failed their clinical endpoints and none have improved long-term patient outcomes. To date, there is no standard of care or FDA-approved drug to treat low/no-reflow after MI.

Thus, there is a present need for improved methods and compositions for treating conditions associated with reperfusion injury and/or low/no-reflow.

### SUMMARY OF THE INVENTION

As described below, the present invention features methods and compositions for treating conditions associated with reperfusion injury, hypofusion, and/or low/no-reflow. In embodiments, the compositions contain complexes containing a basic fibroblast growth factor (FGF2) and an immunoglobulin G (IgG) polypeptide, or fragments thereof. In embodiments, the complexes further contain a hepatocyte growth factor (HGF) polypeptide, a vascular endothelial growth factor (VEGF) polypeptide, or fragments thereof. In embodiments, reperfusion injury, hypofusion, ischemic injury, and/or low/no-reflow is associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. In embodiments, the hypofusion is cerebral hypofusion, tissue hypofusion, and/or organ hypofusion.

In one aspect, the invention features an isolated complex containing a basic fibroblast growth factor (FGF2) polypeptide, or a fragment thereof, and an immunoglobulin G (IgG) polypeptide, or a fragment thereof.

In another aspect, the invention features a composition containing the complex of any of the above aspects.

In another aspect, the invention features a pharmaceutical composition for increasing vascular integrity, promoting angiogenesis, and/or preserving cardiac tissue. The composition contains the complex of any of the above aspects and a pharmaceutically acceptable excipient.

In another aspect, the invention features a method for producing a complex. The method involves contacting an isolated fibroblast growth factor (FGF2) polypeptide or a fragment thereof with an immunoglobulin G (IgG) polypeptide, or a fragment thereof, thereby forming the complex. The method does not involve any concentrating step.

In another aspect, the invention features a method for reducing cell damage or cell death following an ischemic event with reperfusion. The method involves contacting a cell with the complex of any of the above aspects, thereby reducing cell damage or cell death following the ischemic event with reperfusion.

In another aspect, the invention features a method for increasing vascular integrity, promoting angiogenesis, and/or preserving tissue in a subject following an ischemic event with reperfusion. The method involves administering to the subject the complex of any of the above aspects, thereby increasing vascular integrity, promoting angiogenesis, and/or preserving cardiac tissue relative to a reference.

In another aspect, the invention features a method for reducing vascular permeability in a subject following an ischemic event with reperfusion. The method involves administering to the subject the complex of any of the above aspects, thereby reducing vascular permeability relative to a reference.

In any of the above aspects, the complex further contains an additional growth factor polypeptide, or a fragment thereof. In embodiments, the additional growth factor contains a hepatocyte growth factor (HGF) polypeptide, or a fragment thereof. In embodiments, the additional growth factor contains a vascular endothelial growth factor (VEGF) polypeptide, or a fragment thereof.

In any of the above aspects, the polypeptides, or fragments thereof, of the complex are derived from human polypeptides.

In any of the above aspects, the polypeptides are complexed by only non-covalent interactions. In any of the above aspects, the complex does not contain an antibody-antigen interaction.

In any of the above aspects, the ischemic event is associated with reperfusion injury, hypofusion, ischemic injury, and/or no/low-reflow. In embodiments, the hypofusion is tissue and/or organ hypofusion. In embodiments, the hypofusion is cerebral hypofusion. In embodiments, the ischemic event is associated with a burn, diabetic retinopathy, grafted and/or bioengineered tissues, ischemic stroke, ischemic injury, myocardial infarction, organ injury, peripheral artery disease (PAD), sepsis-induced vascular injury, surgery, vascular injury, and/or a wound. In embodiments, the ischemic event is associated with a myocardial infarction. In embodiments, the surgery is an organ transplantation.

In any of the above aspects, cell death occurs during hypoxia associated with the ischemic event.

In any of the above aspects, the contacting or administration occurs within 72 hours of the ischemic event.

In any of the above aspects, the cell is an endothelial cell, smooth muscle cell, fibroblast, cardiac myocyte, skeletal muscle cell, peripheral neuron, CNS neuron, astrocyte, oligodendrocyte, pulmonary epithelial cell, liver epithelial cell, or kidney epithelial cell. In any of the above aspects, the cell is a vascular endothelial cell, a vascular smooth muscle cell, a vascular or cardiac fibroblast, or a cardiac myocyte. In any of the above aspects, the vascular endothelial cell is a microvascular endothelial cell. In any of the above aspects, the cell is a mammalian cell. In any of the above aspects, the cell is a human cell. In any of the above aspects, the cells are in a subject.

In any of the above aspects, the administration is associated with a reduction in vascular permeability relative to a reference. In any of the above aspects, the administration is associated with an increases in vascular integrity. In any of the above aspects, the administration is associated with a reduction in death of cells.

In any of the above aspects or embodiments, the cells contain an endothelial cell, microglial cell, blood-derived cell, smooth muscle cell, fibroblast, cardiac myocyte, skeletal muscle cell, peripheral neuron, CNS neuron, astrocyte, oligodendrocyte, pulmonary epithelial cell, liver epithelial cell, or kidney epithelial cell. In any of the above aspects or embodiments, the cells contain a vascular endothelial cell, a vascular smooth muscle cell, a vascular or cardiac fibroblast, or a cardiac myocyte. In embodiments, the vascular endothelial cell is a microvascular endothelial cell.

The invention provides methods and compositions for treating a condition associated with reperfusion injury, hypofusion, ischemic injury, and/or low/no-reflow. Compositions and articles defined by the invention were isolated or otherwise manufactured in connection with the examples provided below: Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994): The Cambridge Dictionary of Science and Technology (Walker ed., 1988): The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The terms “complex,” “complex of polypeptides,” “protein complex,” or “polypeptide complex” is meant a group of two or more associated polypeptides or fragments thereof. A complex is formed, for example, by two polypeptides that bind, interact or otherwise share some mutual affinity (e.g., electrostatic, hydrophobic, ionic, etc.). In embodiments, the polypeptides are non-covalently associated with one another. In particular embodiments, fibroblast growth factor (FGF2), hepatocyte growth factor (HGF), and/or vascular endothelial growth factor (VEGF), and/or fragments thereof interact with an Fc domain of an IgG or a fragment thereof. In embodiments, the term complex as used herein does not encompass antibody/antigen interactions among IgG and FGF2, HGF, or VEGF. In fact, in some embodiments, antibody/antigen interactions are expressly excluded from the term “complex.”

By “18 kd basic fibroblast growth factor (FGF2) polypeptide” or “basic fibroblast growth factor (FGF2) polypeptide” is meant a polypeptide or fragment thereof comprising an amino acid sequence with at least 85% amino acid sequence identity to GenBank Accession No. AAA52533.1, which is reproduced below, and has mitogenic, angiogenic, and/or neurotrophic activity. An exemplary FGF2 polypeptide is provided below.

(SEQ ID NO: 1)
MAAGSITTLPALPEDGGSGAFPPGHFKDPKRLYCKNGGFFLRIHPDGRV
DGVREKSDPHIKLQLQAEERGVVSIKGVCANRYLAMKEDGRLLASKCVT
DECFFFERLESNNYNTYRSRKYTSWYVALKRTGQYKLGSKTGPGQKAIL
FLPMSAKS.

By “18 kd basic fibroblast growth factor (FGF2) polynucleotide” or “basic fibroblast growth factor (FGF2) polynucleotide” is meant a polynucleotide or fragment thereof encoding FGF2. An exemplary FGF2 polynucleotide is provided at GenBank Accession No. J04513.1, which is reproduced below.

(SEQ ID NO: 2)
ATGGCAGCCGGGAGCATCACCACGCTGCCCGCCTTGCCCGAGGATGGCG
GCAGCGGCGCCTTCCCGCCCGGCCACTTCAAGGACCCCAAGCGGCTGTA
CTGCAAAAACGGGGGCTTCTTCCTGCGCATCCACCCCGACGGCCGAGTT
GACGGGGTCCGGGAGAAGAGCGACCCTCACATCAAGCTACAACTTCAAG
CAGAAGAGAGAGGAGTTGTGTCTATCAAAGGAGTGTGTGCTAACCGTTA
CCTGGCTATGAAGGAAGATGGAAGATTACTGGCTTCTAAATGTGTTACG
GATGAGTGTTTCTTTTTTGAACGATTGGAATCTAATAACTACAATACTT
ACCGGTCAAGGAAATACACCAGTTGGTATGTGGCACTGAAACGAACTGG
GCAGTATAAACTTGGATCCAAAACAGGACCTGGGCAGAAAGCTATACTT
TTTCTTCCAATGTCTGCTAAGAGCTGA.

By “Immunoglobulin G (IgG) polypeptide” is meant an antibody or fragment thereof comprising a heavy chain polypeptide with at least about 85% amino acid sequence identity to GenBank Accession No. AAA02914.1, which is reproduced below, and capable of forming a complex with HGF, FGF2, and/or VEFGA. An exemplary full-length human IgG heavy chain polypeptide is provided below.

(SEQ ID NO: 3)
MDWTWRFLFVVAAATGVQSQMQVVQSGAEVKKPGSSVTVSCKASGGTFS
NYAISWVRQAPGQGLEWMGGIIPLFGTPTYSQNFQGRVTITADKSTSTA
HMELISLRSEDTAVYYCATDRYRQANFDRARVGWFDPWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK.

By “Immunoglobulin G (IgG) polynucleotide” is meant a nucleic acid molecule encoding an IgG polypeptide or fragment thereof. An exemplary polynucleotide encoding a human IgG heavy chain polypeptide is provided at GenBank Accession No. M87789.1, which is reproduced below.

(SEQ ID NO: 4)
ATGGACTGGACCTGGAGGTTCCTCTTTGTGGTGGCAGCAGCTACAGGTG
TCCAGTCCCAGATGCAGGTGGTGCAGTCTGGGGCTGAAGTAAAGAAGCC
TGGGTCCTCGGTGACGGTCTCCTGCAAGGCATCTGGAGGCACCTTCAGC
AACTATGCTATCAGCTGGGTGCGACAGGCCCCTGGACAAGGGCTTGAGT
GGATGGGAGGGATCATCCCTCTTTTTGGTACACCAACCTACTCACAGAA
CTTCCAGGGCAGAGTCACGATTACCGCGGACAAATCCACCAGCACAGCC
CACATGGAGCTGATCAGCCTGAGATCTGAGGACACGGCCGTGTATTACT
GTGCGACAGATCGCTACAGGCAGGCAAATTTTGACCGGGCCCGGGTTGG
CTGGTTCGACCCCTGGGGCCAGGGCACCCTGGTCACCGTCTCCTCAGCC
TCCACCAAGGGCCCATCGGTCTTCCCCCTGGCACCCTCCTCCAAGAGCA
CCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTG
CACACCTTCCCGGCTGTCCTACAGTCCTCAGGACTCTACTCCCTCAGCA
GCGTGGTGACCGTGCCCTCCAGCAGCTTGGGCACCCAGACCTACATCTG
CAACGTGAATCACAAGCCCAGCAACACCAAGGTGGACAAGAAAGTTGAG
CCCAAATCTTGTGACAAAACTCACACATGCCCACCGTGCCCAGCACCTG
AACTCCTGGGGGGACCGTCAGTCTTCCTCTTCCCCCCAAAACCCAAGGA
CACCCTCATGATCTCCCGGACCCCTGAGGTCACATGCGTGGTGGTGGAC
GTGAGCCACGAAGACCCTGAGGTCAAGTTCAACTGGTACGTGGACGGCG
TGGAGGTGCATAATGCCAAGACAAAGCCGCGGGAGGAGCAGTACAACAG
CACGTACCGTGTGGTCAGCGTCCTCACCGTCCTGCACCAGGACTGGCTG
AATGGCAAGGAGTACAAGTGCAAGGTCTCCAACAAAGCCCTCCCAGCCC
CCATCGAGAAAACCATCTCCAAAGCCAAAGGGCAGCCCCGAGAACCACA
GGTGTACACCCTGCCCCCATCCCGGGATGAGCTGACCAAGAACCAGGTC
AGCCTGACCTGCCTGGTCAAAGGCTTCTATCCCAGCGACATCGCCGTGG
AGTGGGAGAGCAATGGGCAGCCGGAGAACAACTACAAGACCACGCCTCC
CGTGCTGGACTCCGACGGCTCCTTCTTCCTCTACAGCAAGCTCACCGTG
GACAAGAGCAGGTGGCAGCAGGGGAACGTCTTCTCATGCTCCGTGATGC
ATGAGGCTCTGCACAACCACTACACGCAGAAGAGCCTCTCCCTGTCTCC
GGGTAAATGA.

In embodiments, the IgG further comprises an immunoglobulin light chain (e.g., an immunoglobulin lambda chain) with at least about 85% amino acid sequence identity to GenBank Accession No. AAA02915.1, which is reproduced below. An exemplary immunoglobulin light chain polypeptide sequence is provided below.

(SEQ ID NO: 5)
MAWALLLLTLLTQDTGSWAQSALTQPASVSGSPGQSITISCTGTNNDVG
SYNLVSWYQQHPGKAPKIMIYEVSKRPSGVSNRFSGSKSGNTASLTISG
LQAEDEADYYCCSYAGSYTVVFGGGTKLTVLGQPKAAPSVTLFPPSSEE
LQANKATLVCLISDFYPGAVTVAWKADSSPVKAGVETTTPSKQSNNKYA
ASSYLSLTPEQWKSHRSYSCQVTHEGSTVEKTVAPTECS.

An exemplary polynucleotide encoding an immunoglobulin light chain is provided at GenBank Accession No. M87790.1, which is reproduced below.

(SEQ ID NO: 6)
ATGGCCTGGGCTCTGCTGCTCCTCACCCTCCTCACTCAGGACACAGGGT
CCTGGGCCCAGTCTGCCCTGACTCAGCCTGCCTCCGTGTCTGGGTCTCC
TGGACAGTCGATCACCATCTCCTGCACTGGAACCAACAATGATGTTGGG
AGTTATAACCTTGTCTCCTGGTACCAGCAGCACCCAGGCAAAGCCCCCA
AAATCATGATTTATGAGGTCAGTAAGCGGCCCTCAGGGGTTTCTAATCG
CTTCTCTGGCTCCAAGTCTGGCAACACGGCCTCCCTGACAATCTCTGGG
CTCCAGGCTGAGGACGAGGCTGATTATTACTGCTGCTCATATGCAGGTA
GTTACACTGTGGTTTTCGGCGGAGGGACCAAACTGACCGTCCTAGGTCA
GCCCAAGGCTGCCCCCTCGGTCACTCTGTTCCCGCCCTCCTCTGAGGAG
CTTCAAGCCAACAAGGCCACACTGGTGTGTCTCATAAGTGACTTCTACC
CGGGAGCCGTGACAGTGGCCTGGAAGGCAGATAGCAGCCCCGTCAAGGC
GGGAGTGGAGACCACCACACCCTCCAAACAAAGCAACAACAAGTACGCG
GCCAGCAGCTATCTGAGCCTGACGCCTGAGCAGTGGAAGTCCCACAGAA
GCTACAGCTGCCAGGTCACGCATGAAGGGAGCACCGTGGAGAAGACAGT
GGCCCCTACAGAATGTTCATAG.

By “Hepatocyte Growth Factor (HGF)” is meant a protein or fragment thereof having at least about 85% identity to NCBI Accession No. NP_000592.3, reproduced below, that binds IgG. In embodiments, the HGF includes a histidine-tag (His-tag) comprising about or at least about 2, 3, 4, 5, 6, 7, 8, 9, or 10 contiguous histidine residues (SEQ ID NO: 7), optionally at the C-terminus or N-terminus of the IgG polypeptide sequence. An exemplary full-length human HGF polypeptide is provided below.

(SEQ ID NO: 8)
MWVTKLLPALLLQHVLLHLLLLPIAIPYAEGQRKRRNTIHEFKKSAKTT
LIKIDPALKIKTKKVNTADQCANRCTRNKGLPFTCKAFVFDKARKQCLW
FPFNSMSSGVKKEFGHEFDLYENKDYIRNCIIGKGRSYKGTVSITKSGI
KCQPWSSMIPHEHSFLPSSYRGKDLQENYCRNPRGEEGGPWCFTSNPEV
RYEVCDIPQCSEVECMTCNGESYRGLMDHTESGKICQRWDHQTPHOURH
KFLPERYPDKGFDDNYCRNPDGQPRPWCYTLDPHTRWEYCAIKTCADNT
MNDTDVPLETTECIQGQGEGYRGTVNTIWNGIPCQRWDSQYPHEHDMTP
ENFKCKDLRENYCRNPDGSESPWCFTTDPNIRVGYCSQIPNCDMSHGQD
CYRGNGKNYMGNLSQTRSGLTCSMWDKNMEDLHRHIFWEPDASKLNENY
CRNPDDDAHGPWCYTGNPLIPWDYCPISRCEGDTTPTIVNLDHPVISCA
KTKQLRVVNGIPTRTNIGWMVSLRYRNKHICGGSLIKESWVLTARQCFP
SRDLKDYEAWLGIHDVHGRGDEKCKQVLNVSQLVYGPEGSDLVLMKLAR
PAVLDDFVSTIDLPNYGCTIPEKTSCSVYGWGYTGLINYDGLLRVAHLY
IMGNEKCSQHHRGKVTLNESEICAGAEKIGSGPCEGDYGGPLVCEQHKM
RMVLGVIVPGRGCAIPNRPGIFVRVAYYAKWIHKIILTYKVPQS.

In one embodiment, an HGF fragment is a 29-35 kDa subunit (e.g., 31, 32, 33 kDa) or a 59-70 kDa subunit (e.g., 63, 64, 65, 66, 67 kDa subunit, Nakamura et al., 1989, Nature 342:440-443).

By “Hepatocyte Growth Factor (HGF) polynucleotide” is meant a nucleic acid molecule encoding an HGF polypeptide or fragment thereof. An exemplary human HGF polynucleotide is provided at NCBI Accession No. NM_000601, which is reproduced below.

(SEQ ID NO: 9)
GGGAGTTCAGACCTAGATCTTTCCAGTTAATCACACAACAAACTTAGCTCATCGCAA
TAAAAAGCAGCTCAGAGCCGACTGGCTCTTTTAGGCACTGACTCCGAACAGGATTCT
TTCACCCAGGCATCTCCTCCAGAGGGATCCGCCAGCCCGTCCAGCAGCACCATGTGG
GTGACCAAACTCCTGCCAGCCCTGCTGCTGCAGCATGTCCTCCTGCATCTCCTCCTGC
TCCCCATCGCCATCCCCTATGCAGAGGGACAAAGGAAAAGAAGAAATACAATTCAT
GAATTCAAAAAATCAGCAAAGACTACCCTAATCAAAATAGATCCAGCACTGAAGAT
AAAAACCAAAAAAGTGAATACTGCAGACCAATGTGCTAATAGATGTACTAGGAATA
AAGGACTTCCATTCACTTGCAAGGCTTTTGTTTTTGATAAAGCAAGAAAACAATGCC
TCTGGTTCCCCTTCAATAGCATGTCAAGTGGAGTGAAAAAAGAATTTGGCCATGAAT
TTGACCTCTATGAAAACAAAGACTACATTAGAAACTGCATCATTGGTAAAGGACGC
AGCTACAAGGGAACAGTATCTATCACTAAGAGTGGCATCAAATGTCAGCCCTGGAG
TTCCATGATACCACACGAACACAGCTTTTTGCCTTCGAGCTATCGGGGTAAAGACCT
ACAGGAAAACTACTGTCGAAATCCTCGAGGGGAAGAAGGGGGACCCTGGTGTTTCA
CAAGCAATCCAGAGGTACGCTACGAAGTCTGTGACATTCCTCAGTGTTCAGAAGTTG
AATGCATGACCTGCAATGGGGAGAGTTATCGAGGTCTCATGGATCATACAGAATCA
GGCAAGATTTGTCAGCGCTGGGATCATCAGACACCACACCGGCACAAATTCTTGCCT
GAAAGATATCCCGACAAGGGCTTTGATGATAATTATTGCCGCAATCCCGATGGCCA
GCCGAGGCCATGGTGCTATACTCTTGACCCTCACACCCGCTGGGAGTACTGTGCAAT
TAAAACATGCGCTGACAATACTATGAATGACACTGATGTTCCTTTGGAAACAACTGA
ATGCATCCAAGGTCAAGGAGAAGGCTACAGGGGCACTGTCAATACCATTTGGAATG
GAATTCCATGTCAGCGTTGGGATTCTCAGTATCCTCACGAGCATGACATGACTCCTG
AAAATTTCAAGTGCAAGGACCTACGAGAAAATTACTGCCGAAATCCAGATGGGTCT
GAATCACCCTGGTGTTTTACCACTGATCCAAACATCCGAGTTGGCTACTGCTCCCAA
ATTCCAAACTGTGATATGTCACATGGACAAGATTGTTATCGTGGGAATGGCAAAAAT
TATATGGGCAACTTATCCCAAACAAGATCTGGACTAACATGTTCAATGTGGGACAA
GAACATGGAAGACTTACATCGTCATATCTTCTGGGAACCAGATGCAAGTAAGCTGA
ATGAGAATTACTGCCGAAATCCAGATGATGATGCTCATGGACCCTGGTGCTACACG
GGAAATCCACTCATTCCTTGGGATTATTGCCCTATTTCTCGTTGTGAAGGTGATACCA
CACCTACAATAGTCAATTTAGACCATCCCGTAATATCTTGTGCCAAAACGAAACAAT
TGCGAGTTGTAAATGGGATTCCAACACGAACAAACATAGGATGGATGGTTAGTTTG
AGATACAGAAATAAACATATCTGCGGAGGATCATTGATAAAGGAGAGTTGGGTTCT
TACTGCACGACAGTGTTTCCCTTCTCGAGACTTGAAAGATTATGAAGCTTGGCTTGG
AATTCATGATGTCCACGGAAGAGGAGATGAGAAATGCAAACAGGTTCTCAATGTTT
CCCAGCTGGTATATGGCCCTGAAGGATCAGATCTGGTTTTAATGAAGCTTGCCAGGC
CTGCTGTCCTGGATGATTTTGTTAGTACGATTGATTTACCTAATTATGGATGCACAAT
TCCTGAAAAGACCAGTTGCAGTGTTTATGGCTGGGGCTACACTGGATTGATCAACTA
TGATGGCCTATTACGAGTGGCACATCTCTATATAATGGGAAATGAGAAATGCAGCC
AGCATCATCGAGGGAAGGTGACTCTGAATGAGTCTGAAATATGTGCTGGGGCTGAA
AAGATTGGATCAGGACCATGTGAGGGGGATTATGGTGGCCCACTTGTTTGTGAGCA
ACATAAAATGAGAATGGTTCTTGGTGTCATTGTTCCTGGTCGTGGATGTGCCATTCC
AAATCGTCCTGGTATTTTTGTCCGAGTAGCATATTATGCAAAATGGATACACAAAAT
TATTTTAACATATAAGGTACCACAGTCATAGCTGAAGTAAGTGTGTCTGAAGCACCC
ACCAATACAACTGTCTTTTACATGAAGATTTCAGAGAATGTGGAATTTAAAATGTCA
CTTACAACAATCCTAAGACAACTACTGGAGAGTCATGTTTGTTGAAATTCTCATTAA
TGTTTATGGGTGTTTTCTGTTGTTTTGTTTGTCAGTGTTATTTTGTCAATGTTGAAGTG
AATTAAGGTACATGCAAGTGTAATAACATATCTCCTGAAGATACTTGAATGGATTAA
AAAAACACACAGGTATATTTGCTGGATGATAAAGATTTCATGGGAAAAAAAATCAA
TTAATCTGTCTAAGCTGCTTTCTGATGTTGGTTTCTTAATAATGAGTAAACCACAAAT
TAAATGTTATTTTAACCTCACCAAAACAATTTATACCTTGTGTCCCTAAATTGTAGCC
CTATATTAAATTATATTACATTTCAAAAAAAAAAAAAAAA.

By “vascular endothelial growth factor (VEGF) polypeptide” or “vascular endothelial growth factor (VEGFA) polypeptide” is meant a polypeptide or fragment thereof comprising an amino acid sequence with at least 85% amino acid sequence identity to NCBI Ref. Seq. Accession No. NP_001020537.2, which is reproduced below, and having angiogenic activity. An exemplary VEGF polypeptide is provided below.

(SEQ ID NO: 10)
MTDRQTDTAPSPSYHLLPGRRRTVDAAASRGQGPEPAPGGGVEGVGARG
VALKLFVQLLGCSRFGGAVVRAGEAEPSGAARSASSGREEPQPEEGEEE
EEKEEERGPQWRLGARKPGSWTGEAAVCADSAPAARAPQALARASGRGG
RVARRGAEESGPPHSPSRRGSASRAGPGRASETMNFLLSWVHWSLALLL
YLHHAKWSQAAPMAEGGGQNHHEVVKFMDVYQRSYCHPIETLVDIFQEY
PDEIEYIFKPSCVPLMRCGGCCNDEGLECVPTEESNITMQIMRIKPHQG
QHIGEMSFLQHNKCECRPKKDRARQEKKSVRGKGKGQKRKRKKSRYKSW
SVYVGARCCLMPWSLPGPHPCGPCSERRKHLFVQDPQTCKCSCKNTDSR
CKARQLELNERTCRCDKPRR.

By “vascular endothelial growth factor (VEGF) polynucleotide” or “vascular endothelial growth factor (VEGFA) polynucleotide” is meant a polynucleotide or fragment thereof encoding VEGF. An exemplary VEGF polynucleotide is provided at NCBI Ref. Seq. Accession No. NM_001025366.3, which is reproduced below. An exemplary VEGF polynucleotide is provided below.

(SEQ ID NO: 11)
CTGACGGACAGACAGACAGACACCGCCCCCAGCCCCAGCTACCACCTCC
TCCCCGGCCGGCGGCGGACAGTGGACGCGGCGGCGAGCCGCGGGCAGGG
GCCGGAGCCCGCGCCCGGAGGCGGGGTGGAGGGGGTCGGGGCTCGCGGC
GTCGCACTGAAACTTTTCGTCCAACTTCTGGGCTGTTCTCGCTTCGGAG
GAGCCGTGGTCCGCGCGGGGGAAGCCGAGCCGAGCGGAGCCGCGAGAAG
TGCTAGCTCGGGCCGGGAGGAGCCGCAGCCGGAGGAGGGGGAGGAGGAA
GAAGAGAAGGAAGAGGAGAGGGGGCCGCAGTGGCGACTCGGCGCTCGGA
AGCCGGGCTCATGGACGGGTGAGGCGGCGGTGTGCGCAGACAGTGCTCC
AGCCGCGCGCGCTCCCCAGGCCCTGGCCCGGGCCTCGGGCCGGGGAGGA
AGAGTAGCTCGCCGAGGCGCCGAGGAGAGCGGGCCGCCCCACAGCCCGA
GCCGGAGAGGGAGCGCGAGCCGCGCCGGCCCCGGTCGGGCCTCCGAAAC
CATGAACTTTCTGCTGTCTTGGGTGCATTGGAGCCTTGCCTTGCTGCTC
TACCTCCACCATGCCAAGTGGTCCCAGGCTGCACCCATGGCAGAAGGAG
GAGGGCAGAATCATCACGAAGTGGTGAAGTTCATGGATGTCTATCAGCG
CAGCTACTGCCATCCAATCGAGACCCTGGTGGACATCTTCCAGGAGTAC
CCTGATGAGATCGAGTACATCTTCAAGCCATCCTGTGTGCCCCTGATGC
GATGCGGGGGCTGCTGCAATGACGAGGGCCTGGAGTGTGTGCCCACTGA
GGAGTCCAACATCACCATGCAGATTATGCGGATCAAACCTCACCAAGGC
CAGCACATAGGAGAGATGAGCTTCCTACAGCACAACAAATGTGAATGCA
GACCAAAGAAAGATAGAGCAAGACAAGAAAAAAAATCAGTTCGAGGAAA
GGGAAAGGGGCAAAAACGAAAGCGCAAGAAATCCCGGTATAAGTCCTGG
AGCGTGTACGTTGGTGCCCGCTGCTGTCTAATGCCCTGGAGCCTCCCTG
GCCCCCATCCCTGTGGGCCTTGCTCAGAGCGGAGAAAGCATTTGTTTGT
ACAAGATCCGCAGACGTGTAAATGTTCCTGCAAAAACACAGACTCGCGT
TGCAAGGCGAGGCAGCTTGAGTTAAACGAACGTACTTGCAGATGTGACA
AGCCGAGGCGGTGA.

By “agent” is meant any small molecule chemical compound, antibody, nucleic acid molecule, polypeptide, polypeptide complex, or fragments thereof. Non-limiting examples of agents include polypeptide complexes, such as those containing FGF2 and IgG, or fragments thereof, optionally where the complexes further contain VEGF, HGF, or fragments thereof.

By “ameliorate” is meant decrease, suppress, attenuate, diminish, arrest, or stabilize the development or progression of a disease. In embodiments, a composition of the invention ameliorates a condition associated with reperfusion injury, hypofusion, ischemic injury, and/or low/no-reflow. In some embodiments, the composition of the invention ameliorates a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), symptoms thereof, and the like. In embodiments, the hypofusion is cerebral hypofusion, tissue hypofusion, and/or organ hypofusion.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. In embodiments, the compositions and methods employ a basic fibroblast growth factor (FGF2), hepatocyte growth factor (HGF), vascular endothelial growth factor (VEGF), and/or IgG analog (e.g., various glycosylated forms including, as a non-limiting example, sialylated forms).

In this disclosure, “comprises,” “comprising.” “containing,” and “having” and the like can have the meaning ascribed to them in U.S. Patent law and can mean “includes,” “including.” and the like: “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected.

By “disease” is meant any condition or disorder that damages or interferes with the normal function of a cell, tissue, or organ. Examples of diseases include any disease or injury that results in a reduction in cell number or biological function, including a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. The disease in embodiments is associated with myocardial infarction (MI), reperfusion injury, hypofusion, ischemic injury, and/or low/no-reflow. Diseases include any ischemic event that causes tissue damage. In embodiments, the hypofusion is cerebral hypofusion, tissue hypofusion, and/or organ hypofusion.

By “effective amount” is meant the amount of an agent required to ameliorate the symptoms of a disease relative to an untreated patient. In one embodiment, the compositions of the invention comprise an effective amount of an isolated and/or purified complex containing FGF2 and IgG, or fragments thereof, optionally where the complex further contains VEGF, HGF, or fragments thereof.

In embodiments, the complexes are used for the therapeutic treatment of ischemic injury. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In embodiments, an effective amount of a composition of the invention contains about or at least about 0.0001, 0.0005, 0.001, 0.0025, 0.005, 0.0075, 0.01, 0.02, 0.03, 0.04, 0.05, 0.06, 0.07, 0.08, 0.09, 0.10, 0.2, 0.3, 0.4, or 0.5 mg of FGF2, HGF, VEGF, and/or IgG per kg of a subject to which the composition is administered. In embodiments, the subject weights 30 kg, 40 kg, 50 kg, 60 kg, 70 kg, 80 kg, 90 kg, 100 kg, 110 kg, 120 kg, 130 kg, 140 kg, 150 kg, 160 kg, 170 kg, 180 kg. 190 kg, or 200 kg.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In one embodiment, an HGF fragment is a 29-35 kDa subunit (e.g., 31, 32, 33 kDa) or a 59-70 kDa subunit (e.g., 63, 64, 65, 66, 67 kDa subunit) as measured by SDS PAGE.

By “hypofusion” is meant a reduced amount of blood flow. In embodiments, hypofusion is distinct from frank ischemia or complete block of flow. In embodiments, the reduced amount of blood flow is relative to a reference. In some instances, ischemia is associated with hypofusion. Non-limiting examples of hypofusion include cerebral hypofusion, tissue hypofusion, and/or organ hypofusion.

By “increase” is meant to alter positively by at least 5%. An increase may be by 5%, 10%, 25%, 30%, 50%, 75%, or even by 100%.

The terms “isolated,” “purified.” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector: into an autonomously replicating plasmid or virus; or into the genomic DNA of a prokaryote or eukaryote: or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis. When a cellular factor is “isolated” from a cultured epicardial progenitor cell the cellular factor is typically separated from cells and cellular debris. It need not be purified to homogeneity. In fact, the composition comprising an isolated cellular factor typically comprises any number of cellular factors whose presence contributes to the biological activity (e.g., growth promoting, survival promoting, or proliferation promoting activity) of the composition. In one embodiment, a composition of the invention comprises or consists of conditioned media from which cells and cellular debris have been removed.

By “marker” is meant any protein or polynucleotide having an alteration in expression level or activity that is associated with a developmental state, condition, disease, or disorder. A non-limiting example of a marker of plasma injury (e.g., vascular injury after ST segment elevation myocardial infarction (STEMI)) is Angiopoietin-2 (Angpt-2).

As used herein, “obtaining” as in “obtaining an agent” includes synthesizing, purchasing, or otherwise acquiring the agent.

As used herein, the terms “prevent.” “preventing.” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant a negative alteration of at least 10%, 25%, 50%, 75%, or 100%. In particular, vascular permeability may be reduced relative to a reference (e.g., untreated control vessel) by at least about 5%, 10%, 25%, 30%, 50%, 75%, or even by 100%.

By “reference” is meant a standard or control condition. Non-limiting examples of a reference include a subject not having a disease, a healthy cell or subject, or a cell or subject not exposed to a particular stress (e.g., low oxygen stress, low/no-reflow, reperfusion, hypofusion, ischemic injury, and/or myocardial infarction (MI)), or an untreated corresponding control subject (e.g., having an untreated MI, hypofusion, ischemic injury, or reperfusion injury).

A “reference sequence” is a defined sequence used as a basis for sequence comparison. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example. Sequence Analysis Software Package of the Genetics Computer Group. University of Wisconsin Biotechnology Center. 1710 University Avenue, Madison, Wis. 53705. BLAST. BESTFIT. GAP, or PILEUP/PRETTYBOX programs). Such software matches identical or similar sequences by assigning degrees of homology to various substitutions, deletions, and/or other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “repair” is meant to ameliorate damage or disease in a tissue or organ.

By “tissue” is meant a collection of cells having a similar morphology and function.

As used herein, the terms “treat,” treating.” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “vascular integrity” is meant maintenance of flow in a vessel without leakage. Exemplary vessels include arteries, arterioles, veins, venules, capillaries, and microvessels. The flow in embodiments is fluid flow or flow of cells. In embodiments, the leakage is leakage of a fluid and/or cells.

By “vascular permeability” is meant leakage from a vessel into the surrounding environment. The leakage in embodiments is of blood or another bodily fluid.

By “subject” is meant an animal. The animal can be a mammal. The mammal can be a human or non-human mammal, such as a bovine, equine, canine, ovine, rodent, or feline.

As used herein, the terms “treat,” treating.” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”. “an”, and “the” are understood to be singular or plural.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a plot showing a correlation between infarct size and plasma Angpt-2 levels in patients at 48 hrs after STEMI. For linear regression, data points for Angpt-2 represent area under the 48 hr plasma Angpt-2/time curve.

FIGS. 2A and 2B provide images and a bar graph showing intracoronary infusion of the HGF/IgG complex from percutaneous coronary intervention (PCI) guide catheter salvages myocardium at risk after myocardial infarction (MI). The leftmost images in FIG. 2A show Evan's blue dye stain showing areas with adequate perfusion (generally the lower areas in the images) and low perfusion (generally the upper areas in the images; a.k.a. Area At Risk, AAR). The rightmost images in FIG. 2A are triphenyltetrazolium chloride (TTC) stain images showing living muscle tissue (dark areas) and necrotic, dying tissue (lighter areas; a.k.a. Ischemic Area. IA). Note: For HGF/IgG-treated pig (bottom), the yellow dashed lines delineate regions of muscle tissue with low perfusion that remain alive. FIG. 2B provides a bar graph showing that, combined with primary PCI. HGF/IgG complex infusion rescued 48±12% tissue at risk at 24 hrs after MI. N=4 to 8 adult female swine (˜50 kg). Myocardial salvage index=AAR-IA/AAR.

FIG. 3 provides a plot demonstrating that multiple VasaPlex treatments with HGF/IgG did not elicit anti-drug antibodies (ADA). Study design; Day 0; All rats. 2 hrs ischemia w/reperfusion. After myocardial infarction (MI), treatments were: day 0 (intra-cardiac, upon reperfusion), and days 3 and 5 (IV, tail vein). Control=vehicle. Blood plasma was sampled weekly for enzyme-linked immunosorbent assay (ELISA). N=4-5 rats/group.

FIG. 4 provides a gel image demonstrating that FGF2 seeds spontaneous formation of FGF2:HGF:IgG complexes without the need for ultracentrifgual units/centrifugation. A native isoeletric focusing gel was used to detect protein complexes. Lanes: 1) IgG alone: 2) FGF2 alone: 3) FGF2:IgG complex: 4) HGF alone: 5) free HGF+free IgG (no complex formed; note two separate bands): 6) Spontaneous complex formed with FGF2:HGF:IgG in PBS (pH 7.4), at room temp.

FIG. 5 provides a gel image demonstrating that heparin prevented FGF2:IgG complexation and disassociated pre-existing FGF2:IgG complexes. A native isoeletric focusing gel was used to detect protein complexes. Lanes: 1) IgG alone: 2) FGF2 alone: 3) FGF2:IgG complex: 4) Mixing heparin with free FGF2 and free IgG prevented complexation: 5) Addition of heparin to pre-existing FGF2:IgG complexes dissociated them (compare lane 3 to lane 5).

FIG. 6 presents a bar graph demonstrating protection of primary human endothelial cells by the indicated complexes. ** P<0.001.

FIGS. 7A-7C present bar graphs showing results from biochemical pulldown assays with protein A-sepharose and streptavidin-agarose for analysis of protein complexes. FIGS. 7A and 7B present bar graphs presenting results from an experiment where pre-formed complexes were incubated with protein A-sepharose and centrifuged to isolate intact complexes. FIG. 7C presents a bar graph presenting results from pulldowns with biotinylated antibodies to the His-tag on a recombinant hepatocyte growth factor (HGF) confirmed that basic fibroblast growth factor (FGF2) and HGF were in the same complex. Pellets for FIGS. 7A-7C were washed with phosphate buffered saline (PBS) and solubilized with 1% sodium deoxycholate prior to enzyme-linked immunosorbent assays (ELISAs) to measure HGF or FGF2 levels.

FIG. 8 presents a bar graph showing electrode recordings from CoreMap's array detect functional myocardial tissue within the infarcted region at 24 hrs after myocardial infarction (MI). N=120 electrodes. Note: Readings below 1.5 mV showed that 17% of myocytes were non-functioning in the area recorded, illustrating that measurements were made within a zone with infarction.

FIG. 9 provides a gel image demonstrating that basic fibroblast growth factor (FGF2) seeds formation of FGF2:IgG. FGF2:VEGF:IgG and FGF2:HGF:IgG complexes without the need for ultracentrifgual units/centrifugation. A native isoeletric focusing gel was used to detect protein complexes. Lanes: 1) IgG alone: 2) basic fibroblast growth factor (FGF2) alone: 3) FGF2:IgG complex: 4) HGF alone: 5) Free hepatocyte growth factor (HGF)+free IgG: note two distinct bands: 6) FGF2:HGF:IgG complexes formed in PBS. pH 7.5: 7) VEGF alone: 8) VEGF+IgG. Compare with IgG alone in lane 1: 9) VEGF+FGF2: 10) FGF2:VEGF:IgG complex. Simple mixing of FGF2 with IgG or with HGF and IgG resulted in mobility shifts indicative of complex formation. The gel was run at room temperature and stained for 30 min with Coomassie brilliant blue and de-stained overnight in 10% acetic acid.

FIG. 10 presents bar graphs showing pulldown of FGF2:HGF:IgG complexes by protein-A sepharose. Pre-formed complexes composed of human FGF2, human HGF and porcine IgG were incubated with Protein A-Sepharose beads. Pelleted beads were solubilized with 1% sodium deoxycholate to liberate bound factors prior to enzyme-linked immunosorbent assays (ELISAs). Left panel of FIG. 10: ELISA for human FGF2 revealed significantly greater FGF2 signal within the detergent soluble pulldown fraction, compared with the pulldown supernatant. (N=3 replicates). Right panel of FIG. 10: ELISA for human HGF showed significantly greater HGF signal within the detergent soluble fraction pulldown fraction, compared with pulldown supernatant. (N=3 replicates). Data are mean±SD. Unpaired Student's t-test for PD fraction vs supernatant. * P≤0.05. Throughout the figures. PD indicates “pulldown”.

FIG. 11 presents bar graphs showing pulldown of FGF2:HGF:IgG complexes by anti-6× histidine biotin (SEQ ID NO: 12) and streptavidin agarose. Pre-formed FGF2:HGF:IgG complexes were incubated with biotinylated antibodies to 6×His-tag (SEQ ID NO: 12) on HGF and pulled down with streptavidin-agarose. Left panel of FIG. 11: ELISA for human FGF2 reveals positive signal detected within detergent soluble pulldown fraction. Right panel of FIG. 11: ELISA for human FGF2 reveals signal within detergent soluble pulldown fraction. As basic fibroblast growth factor (FGF2) lacks a 6×-his tag (SEQ ID NO: 12), any positive signal in the detergent soluble fraction from streptavidin pulldown is indicative of interaction between FGF:HGF:IgG. N=1 assay. PD=pulldown.

FIG. 12 presents a gel image demonstrating that heparin prevents FGF2:IgG and FGF:HGF:IgG complex formation and disassociates pre-existing FGF2:IgG complexes. A native isoeletric focusing gel was used to detect protein complexes. Lanes: 1) IgG alone: 2) FGF2 alone: 3) FGF2:IgG complex: 4) Mixing heparin with free FGF2 and free IgG prevented complexation: 5) Addition of heparin to pre-existing FGF2:IgG complexes dissociated them (compare lane 3 to lane 5).

FIG. 13 provides two bar graphs demonstrating liberation of growth factors from FGF2:HGF:IgG complexes by co-incubation with heparin. Pre-formed complexes containing human basic fibroblast growth factor (FGF2), rat hepatocyte growth factor (HGF), and mixed porcine IgG were incubated with protein A-sepharose beads for pulldown assays. Centrifuged and pelleted beads were solubilized with 1% sodium deoxycholate to liberate bound factors prior to enzyme-linked immunosorbent assays (ELISAs) to detect growth factors. Left panel of FIG. 13: Enzyme-linked immunosorbent assay (ELISA) for human basic fibroblast growth factor (FGF2) showed significantly greater FGF2 signal within the detergent soluble pulldown fraction, compared with the supernatant. (N=3 assays). Right panel of FIG. 13: Pre-formed complexes containing human FGF2, human HGF and mixed porcine IgG were incubated with Protein A-Sepharose beads. Centrifuged and pelleted beads were solubilized with 1% sodium deoxycholate to liberate bound factors prior to ELISAs. Results indicated greater hepatocyte growth factor (HGF) signal within the detergent soluble pulldown fraction, compared with the pulldown supernatant. (N=1 assay). Unpaired Student's t-test. PD fraction vs. FGF+HGF control (No IgG). ** P<0.001. PD=pulldown.

FIG. 14 provides images and a bar graph demonstrating that intracoronary delivery of FGF2:HGF:IgG complexes preserved jeopardized myocardium after myocardial infarction (MI) with reperfusion. Left panel of FIG. 14: Evan's blue dye stain shows areas with adequate perfusion (generally the upper portions of the images, including the lighter areas) and low perfusion (generally the lower portions of the images, including the darker areas). Right panel of FIG. 14: TTC stain shows living muscle tissue (darker areas) and necrotic, dying tissue (lighter areas). Note: For FGF2:HGF:IgG-treated pig (left, bottom), Pigs were treated with 12.5 μg of FGF2, 63 μg HGF and 210 μg mixed polyclonal IgG; representing a 1:1:2 molar ratio. Infusion of FGF2:HGF:IgG complexes rescued (avg) 37.67% of tissue at risk at 24 hours after myocardial infarction (MI) (FGF2:HGF:IgG. N=2). (DMEM vehicle control. N=4). Myocardial salvage index=AAR-IA/AAR. Unpaired Student's t-test. * P<0.05.

FIGS. 15A-15C provide bar graphs and an image relating to unipolar electrophysiological recordings using the CoreMap high density electrode array. For each animal, a reference recording was taken from healthy non-infarcted tissue, serving as an internal control (Norm). The reference voltage was used to calculate the difference in voltage % from the infarcted region of left ventricle (LV) (Inf). The voltage % reduction in the FGF2:HGF:IgG treated pigs, relative to the vehicle treated pigs, was representative of a larger volume of preserved myocardium within the recording region. FIG. 15C provides an image showing the CoreMap high density electrode array. Vehicle. N=4 pigs; VasaPlex-F2. N=3 pigs. Student's t-test (unpaired). ** P=0.037.

FIGS. 16A and 16B present bar graphs relating to pulldown of FGF2:HGF:IgG complexes by protein-A sepharose. Pre-formed complexes containing human basic fibroblast growth factor (FGF2), rat hepatocyte growth factor (HGF) and mixed porcine IgG were incubated with protein A-sepharose beads. Pelleted beads were solubilized with 1% sodium deoxycholate to liberate bound factors prior to enzyme-linked immunosorbent assays (ELISAs). FIG. 16A provides a bar graph showing results from ELISA for human FGF2. The results demonstrated significantly greater FGF2 signal within the detergent soluble pull-down fraction, compared with the FGF2+HGF control (No IgG). FIG. 16B provides a bar graph showing results from ELISA for rat HGF. The results demonstrated significantly greater HGF within the detergent soluble pull-down fraction, compared with the FGF2+HGF control (No IgG). The signal detected by ELISA indicated specific interaction of complexes with protein-A agarose; i.e., successful pulldown (N=3 assays). Data are mean±SD. Unpaired Student's t-test PD fraction vs. FGF2+HGF control (No IgG). **** P<0.0001. *** P<0.001. PD=pull down.

FIGS. 17A-17D present schematics and protein structural images showing three-dimensional modeling of proposed growth factor:immunoglobulin complexes. Top panel of FIG. 17A: Schematic representations of full-length FGF2 and FGFR1. Bottom panel of FIG. 17A: Ribbon representations of FGF2 (residues 158-286) bound to FGFR1 and heparin. Note the binding of FGF2 to FGFR1 occurred within immunoglobulin-like sub-domains. Top panel of FIG. 17B: Schematic representations of the proposed binding of FGF2 to the IgG1 Fc domain. Bottom panel of FIG. 17B: Ribbon representation of proposed binding of FGF2 to the Fc domain of IgG within FGF2:IgG complexes. Based on similarity to FGF2:FGFR1 binding, it is possible the Fc domain of IgG may accommodate the binding of two FGF2 ligands. Top panel of FIG. 17C: Schematic representations of the HGF pan domain and IgG1. Bottom panel of FIG. 17C: Ribbon representation of proposed binding of HGF pan domain to the Fc domain of IgG1 in HGF:IgG complexes. In FIGS. 17A-17C, the hexagon (top panels) and sticks (bottom panels) represent glycosylation (e.g., with heparin). FIG. 17D: Electrostatic surface potential models of FGF2 and HGF pan domain bound to heparin (sticks). Note growth factors have heparin binding domains that are thought to bind within immunoglobulin-like domains of their respective RTK receptors. Basic patches (darker shading) may be involved in the binding of these growth factors in our complexes. PK=protein kinase. HC=heavy chain. K1-K4=Kringle domains. SPH=serine protease homology domain, alpha=HGF alpha chain, beta=HGF beta chain. Gray=domains not shown in ribbon diagrams.

FIGS. 18A-18B provide schematics and protein ribbon structures showing three-dimensional modeling of proposed FGF2:HGF:IgG complexes: FIG. 18A provides a schematic representation of whole molecule IgG glycosylated (hexagon). FGF2 (residues 158-286) and HGF pan domain. FIG. 18B provides a ribbon representations of proposed FGF2. HGF. IgG binding that occurs within FGF2:HGF:IgG complexes. Note this model was generated by overlaying FGF2:IgG Fc and HGF pan:IgG Fc models with full-length IgG (PDB ID: 1HZH). Individual docking of FGF2 or HGF pan with full-length IgG returned results similar to Fc docking experiments showing many high scoring poses of FGF2 and HGF interacting between the CH2 and CH3 domains in the Fc. HC=heavy chain. LC=light chain. K1-K4=Kringle domains. SPH=serine protease homology domain, alpha=HGF alpha chain, beta=HGF beta chain. The K1, K2, K3, K4, and SPH domains are not shown in ribbon diagrams.

FIG. 19 provides a bar graph demonstrating that FGF2/HGF/IgG and FGF2:HGF:IgG complexes protected human cardiac microvascular endothelial cells against simulated ischemia. The commercially available Cy Quant assay demonstrated protection of endothelial cells conferred by FGF/HGF/IgG (Amicon-concentrated) complexes and FGF2:HGF:IgG (spontaneous formation) complexes under conditions simulating ischemia (1% oxygen and nutrient deprivation). N=3 human donors. By one-way ANOVA: FGF2/HGF/IgG compared with DMEM control. * P=0.0169; FGF2:HGF:IgG compared with DMEM. ** p=0.0063.

### DETAILED DESCRIPTION OF THE INVENTION

The invention features, among other things, methods and compositions for treating a condition associated with reperfusion injury, hypofusion, ischemic injury, and/or low/no-reflow. In embodiments, the composition contain complexes containing a basic fibroblast growth factor (FGF2) polypeptide and an immunoglobulin G (IgG) polypeptide, or fragments thereof, optionally where the complexes further contain a hepatocyte growth factor (HGF) polypeptide, a vascular endothelial growth factor (VEGF) polypeptide, or fragments thereof.

In instances, the compositions are vaso- and/or cardioprotective and/or associated with an increase in vascular integrity and/or preservation of tissue (e.g., cardiac tissue) jeopardized by reperfusion injury, hypofusion, ischemic injury, and/or low/no re-flow. Compositions of the present invention reduce infarct size and improve patient outcomes after myocardial infarction. In embodiments, reperfusion injury, hypofusion, ischemic injury, and/or low/no-reflow is associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. In embodiments, the hypofusion is cerebral hypofusion, tissue hypofusion, and/or organ hypofusion.

The present invention is based, at least in part, upon the discovery that complexes comprising FGF2 and IgG can form spontaneously, and the discovery that complexes containing FGF2. HGF, and IgG protect human microvascular endothelial cells against simulated ischemia, and preserve functional myocardial tissue subsequent to myocardial infarction (MI). As presented in the Examples provided herein, by mixing FGF2 with HGF and IgG at a molar ratio of 1:1:2. FGF2 seeded complex formation with HGF and IgG in the absence of any concentrating step (e.g., a centrifugation method).

The combination of FGF2 and HGF is angiogenic (i.e., promotes blood vessel sprouting from pre-existing vessels). Moreover, in embodiments, the combination of FGF2 and HGF is associated with generation of stable vessels that last longer than those formed with HGF alone. The complexes of the present invention (e.g., complexes containing FGF2 and IgG, or fragments thereof, optionally where the complexes further contain VEGF, HGF, or fragments thereof) can be easily produced in a clinical setting from FGF2. HGF, and IgG. The complex containing FGF2 and IgG forms a basic biochemical infrastructure from which to build custom agonist or antagonist signaling complexes with ligands of desired properties, for particular indications (e.g., complexes containing FGF2 and IgG, or fragments thereof, optionally where the complexes further contain VEGF, HGF, or fragments thereof). This biochemical infrastructure is likely to allow for the preparation of custom complexes constituting designer biologic drugs that target particular biological processes to obtain a desired outcome.

Accordingly, the invention provides complexes comprising FGF2, HGF, and IgG, and methods of using such complexes to treat a variety of indications associated with hypofusion and/or ischemic injury (e.g., reperfusion injury, hypofusion, and/or low/no-reflow associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like). In embodiments, the hypofusion is cerebral hypofusion, tissue hypofusion, and/or organ hypofusion.

### Reperfusion Injury and Low/No-Reflow

Reperfusion injury and low/no-reflow after myocardial infarction and percutaneous coronary intervention (PCI) increase infarct size and mortality. Not being limited by theory, microvascular integrity and extent of no/low-reflow are determinants of infarct expansion. Consequently, numerous mechanical (e.g. thrombectomy) and pharmacological approaches (e.g. adenosine, nitroprusside, nicorandil, verapamil) have been tested to prevent or alleviate no/low-reflow. The 5 year prognosis for mortality in no-reflow patients with acute ST segment elevation myocardial infarction (STEMI) remains poor relative to those with reflow, and all phase III clinical trials have failed to provide long-term benefit. Among a group of 1.406 patients with ST segment elevation myocardial infarction (STEMI) that underwent percutaneous coronary intervention (PCI), 410 (29%) were diagnosed with no-reflow (Ndrepepa et al., J. Am. Coll. Cardiol. 55, 2383-2389 (2010)). Kaplan-Meier estimates of 5-year mortality were 18.2% for patients with no-reflow and 9.5% for reflow patients. Infarct size was highly correlated to incidence of no-reflow. The mean infarct size in no-reflow patients was 15.0% of the left ventricle whereas in reflow patients it was 8% of the left ventricle. In addition to increased infarct size, patients with no/low-reflow have a higher incidence of early post-infarction complications (e.g. arrhythmias, pericardial effusion, early congestive heart failure), and adverse left ventricular remodeling compared with those with reflow.

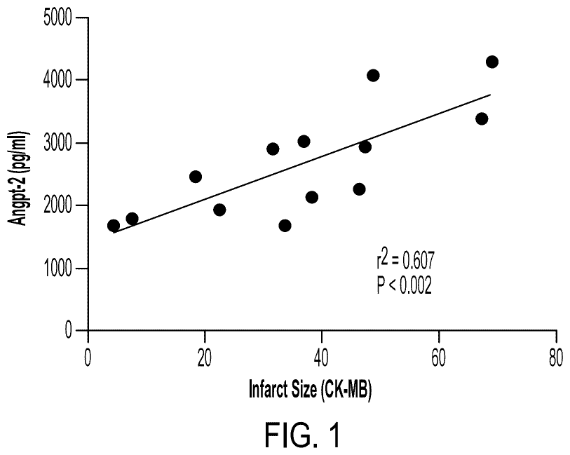

Angiopoietin-2 (Angpt-2) is a potential diagnostic plasma marker for vascular injury after ST segment elevation myocardial infarction (STEMI) (Tarikuz Zaman A K M. French C J. Spees J L. Binbrek A S. Sobel B E. “Vascular rhexis in mice subjected to non-sustained myocardial ischemia and its therapeutic implications” Exp. Biol. Med. (Maywood) 236:598-603 (2011)). Not being bound by theory. Angpt-2 is released into the circulation in a bi-phasic pattern after myocardial infarction (MI): first from necrotic endothelial cells early after ischemia/reperfusion and later during angiogenesis for tissue repair. Creatine kinase is an enzyme released from necrotic cardiac myocytes after MI. Notably, at 48 hr after MI, plasma Angpt-2 levels in patients were are correlated to infarct size determined by analysis of circulating creatine kinase activity (MB isoform)(P=0.0017. FIG. 1) (Tarikuz Zaman A K M. French C J. Spees J L. Binbrek A S. Sobel BE. “Vascular rhexis in mice subjected to non-sustained myocardial ischemia and its therapeutic implications” Exp. Biol. Med. (Maywood) 236:598-603 (2011)). Circulating Angpt-2 levels correlate to peak levels of cardiac troponin T, a marker widely used to estimate infarct size. Thus, endothelial cell/vascular injury is a predictor of infarct expansion/size and a potential therapeutic target.

As reported in detail below: complexes containing FGF2, HGF, and IgG were found to reduce reperfusion injury after myocardial infarction and percutaneous coronary intervention (PCI).

### Cardioprotective Treatments and PCI

Primary percutaneous coronary intervention (PCI) is the Standard of Care (SoC) for ST segment elevation myocardial infarction (STEMI). Typically, the percutaneous coronary intervention (PCI) guide catheter is removed immediately after stenting. However, this means that a valuable opportunity is missed to directly treat the affected arteries, arterioles, and capillaries downstream of the occlusive site. Because up to 50% of final infarct size in patients is determined by reperfusion injury and the degree of low/no-reflow after myocardial infarction (MI) and percutaneous coronary intervention (PCI), treatment strategies that are vaso-protective and/or angiogenic (e.g., complexes containing FGF2 and IgG, or fragments thereof, optionally where the complexes further contain VEGF, HGF, or fragments thereof) have great potential to reduce or prevent infarct expansion, decrease final infarct size, and improve patient outcomes. In embodiments, a complex containing FGF2 and IgG, or fragments thereof, optionally where the complexes further contain VEGF, HGF, or fragments thereof, is administered to a subject as biologic drug treatments integrated into the PCI procedure. Because ischemic tissue injury increases over time, a major concern with cardioprotective treatments is whether or not they increase time to stenting and reperfusion. Importantly, compositions of the present invention (e.g., compositions containing complexes containing FGF2 and IgG, or fragments thereof, optionally where the complexes further contain VEGF, HGF, or fragments thereof) can be delivered directly into the cardiac circulation through the indwelling PCI guide catheter—after/during stenting and/or before/after reperfusion. In embodiments, the compositions of the present disclosure are safe, well tolerated, and require only a limited time (e.g., 1, 2, 3, 4, 5, 6, 7, 8, 9, 10, 15, or 20 minutes) to infuse prior to removal of the guide catheter.

While about 30 to about 50% of patients exhibit low/no-reflow after myocardial infarction (MI), all patients undergoing percutaneous coronary intervention (PCI) have reperfusion injury that could be treated. Combining administration of compositions of the present invention (e.g., compositions containing complexes containing FGF2 and IgG, or fragments thereof, optionally where the complexes further contain VEGF, HGF, or fragments thereof) with primary PCI as standard of care could benefit over 800,000 Americans each year and millions of myocardial infarction (MI) patients worldwide. Given the rapidly growing global market for cardiovascular disease, agents of the present disclosure are likely to be game changing, blockbuster drugs.

### Complexes

The present invention provides agents comprising complexes containing basic fibroblast growth factor (FGF2), hepatocyte growth factor (HGF), vascular endothelial growth factor (VEGF), and/or IgG (e.g., complexes containing FGF2 and IgG, or fragments thereof, optionally where the complexes further contain VEGF, HGF, or fragments thereof). In embodiments, the complexes are self-assembling (i.e., they form spontaneously in solution after addition of FGF2). Not being bound by theory. FGF2 seeds complex formation. In embodiments, the complexes are formed in a method that does not involve a concentrating and/or centrifugation step. Various complexes of the present invention are formed without the need for any concentration and/or centrifugation step, although preformed complexes can optionally be concentrated using various methods (e.g., ultracentrifugation).

Not being bound by theory, the heparin-mimetic binding activity in the Immunoglobulin G (IgG) Fc domain forms therapeutic protein complexes with angiogenic, heparin-binding growth factors such as hepatocyte growth factor (HGF) and basic Fibroblast Growth Factor (FGF2). Mammalian IgG molecules possess N-glycosylation sites in the Fc domain that affect their function(s). The sugar molecules located at these sites can be further modified by fucosylation, galactosylation, and sialylation. Not wishing to be bound by theory, heparin molecules are negatively-charged polysaccharides that promote the formation of anti-thrombin:thrombin protein complexes. These complexes deactivate thrombin and prevent blood coagulation. Due to over-expansion of B cell clones that express particular IgG glycoforms, many cancer patients with multiple myeloma present with bleeding complications, in part, due to formation of IgG1: anti-thrombin complexes that inhibit thrombin, thereby increasing time to clot (similar in effect to addition of heparin). Not being bound by theory, the agents of the present invention may perform by a similar mechanism. i.e. the glycosylated, negatively-charged Fc domain of IgG may attract the heparin-binding domains of HGF and FGF2.

In embodiments, purified recombinant FGF2, HGF, and/or VEGF is combined with IgG isolated and/or purified from human sources or other mammalian sources (i.e. rat, mouse, rabbit, pig, goat), or recombinant IgG. In this manner, complexes (e.g., complexes containing FGF2 and IgG, or fragments thereof, optionally where the complexes further contain VEGF, HGF, or fragments thereof) may be assembled by various means. Non-limiting examples of such means include spontaneous formation by contacting two polypeptides with one another (i.e., with no concentration step), with concentration by filtration, with centrifugation, column chromatography, changes in temperature or density, or by effectively increasing concentration through addition of particular molecules such as dextran sulphate or polyethylene glycol as is standard in the art in methods associated with developing probes for in situ hybridization.

Alternatively, more simple means of forming complexes may be employed such as through altering the effective concentrations of polypeptides forming the complex (e.g., FGF2, HGF, VEGF, and/or IgG).

In embodiments, the complexes (e.g., complexes containing FGF2 and IgG, or fragments thereof, optionally where the complexes further contain VEGF, HGF, or fragments thereof) provide enhanced vaso-protection compared with free (i.e., non-complexed) fibroblast growth factor (FGF2), or free hepatocyte growth factor (HGF), alone or in combination. The methods and compositions of the present invention preserve vascular integrity and improve cardiac function. Not being bound by theory, the complexes are associated with a reduction in endothelial cell injury and vascular permeability. In embodiments, the complexes are associated with an increase in endothelial cell survival. In some instances, the complexes are associated with activation (e.g., via phosphorylation) of c-Met (an HGF receptor). Ryk (a Wingless (Wnt) co-receptor: also known as “related to tyrosine kinase”), and/or FGFR. Complex formation increases the half-life of FGF2. VEGF, and/or HGF relative to free FGF2, free VEGF, or free HGF.

Since the complexes containing FGF2 and IGF form spontaneously in buffered saline solution without the need for centrifugation, fresh complexes (e.g., complexes containing FGF2 and IgG, or fragments thereof, optionally where the complexes further contain VEGF, HGF, or fragments thereof) could be generated in the clinical setting without the need for specialized equipment and by comparatively simple methods (e.g., methods involving less steps). In addition, while HGF alone induces angiogenesis, the combination of HGF and FGF2 generates more durable capillaries and microvasculature. Thus, whereas complexes containing HGF and IgG and complexes containing FGF2, HGF, and IgG combined each provide vaso-protection, cardio-protection and angiogenesis, the complexes containing FGF2, HGF, and IgG provide additional benefit(s) for patients in terms of durable blood vessel growth, myocardial perfusion and cardiac function.

The complexes of the invention comprise IgG in complex with an additional polypeptide(s) (e.g., FGF2, HGF, and/or VEGF). In embodiments, a composition of the invention comprise IgG in an amount such that the molar ratio of IgG to the additional polypeptide(s) in the composition is about or at least about 0.5, 1, 1.5, 2, 2.5, 3, 3.5, 4, or 5. In some instances, the compositions comprise equimolar amounts of the additional polypeptides.

In embodiments, complexes containing FGF2, HGF, and IgG are at least as vaso- and cardioprotective as complexes containing HGF and IgG.

### Polypeptide Production

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen. Inc., Madison. Wis). According to this expression system. DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3× may be cleaved with factor Xa.

Once a recombinant polypeptide of the invention is expressed, it is isolated. e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see. e.g., Ausubel et al., supra). Once isolated, the recombinant protein can, if desired, be further purified. e.g., by high performance liquid chromatography (see. e.g., Fisher. Laboratory Techniques In Biochemistry and Molecular Biology, eds., Work and Burdon. Elsevier. 1980). Polypeptides of the invention, particularly short peptide fragments, can also be produced by chemical synthesis (e.g., by the methods described in Solid Phase Peptide Synthesis. 2nd ed., 1984 The Pierce Chemical Co., Rockford. Ill.). These general techniques of polypeptide expression and purification can also be used to produce and isolate useful peptide fragments or analogs.

### Therapeutic Methods

Compositions comprising complexes comprising FGF2, IgG, HGF, and VEGF or other growth factors are useful for preventing or ameliorating tissue damage associated with reperfusion injury, hypofusion, ischemic injury, and/or low/no-reflow (e.g., a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like). In embodiments, the hypofusion is cerebral hypofusion, tissue hypofusion, and/or organ hypofusion. In one therapeutic approach, an isolated complex containing FGF2 and IgG, or fragments thereof, optionally where the complex further contains VEGF, HGF, or fragments thereof, is administered systemically. The dosage of the administered isolated complex depends on a number of factors, including the size and health of the individual patient. For any particular subject, the specific dosage regimes should be adjusted, as necessary over time according to the individual need and the professional judgment of the person administering or supervising the administration of the compositions.

In embodiments, the composition is administered to a subject following myocardial ischemia with reperfusion. In embodiments, the composition is administered within about 1 hr. 2 hrs, 3 hrs, 4 hrs, 5 hrs, 6 hrs, 12 hrs, 24 hrs, 48 hrs, 72 hrs, 1 week, 2 weeks, 3 weeks, or 1 month of myocardial ischemia with reperfusion. In embodiments, the composition is administered within about 1 hr. 2 hrs, 3 hrs, 4 hrs. 5 hrs, 6 hrs, 12 hrs. 24 hrs, 48 hrs, 72 hrs, 1 week, 2 weeks, 3 weeks, or 1 month of a myocardial infarction. In embodiments the administration is within the indicated periods before and/or after myocardial ischemia with reperfusion or a myocardial infarction.

### Pharmaceutical Compositions

In one embodiment, a composition of the invention comprises or consists essentially of isolated complexes containing FGF2 and IgG, or fragments thereof, optionally where the complexes further contain VEGF, HGF, or fragments thereof. An isolated complex can be conveniently provided to a subject as sterile liquid preparations, e.g., isotonic aqueous solutions, suspensions, emulsions, dispersions, or viscous compositions, which may be buffered to a selected pH. A composition comprising isolated complexes containing FGF2 and IgG, or fragments thereof, optionally where the complexes further contain VEGF, HGF, or fragments thereof, may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by mixing isolated complexes containing FGF2 and IgG, or fragments thereof, optionally where the complexes further contain VEGF, HGF, or fragments thereof, in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose). pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”. 17th edition. 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic. i.e., they can have the same osmotic pressure as blood and/or lachrymal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form. e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

Compositions comprising isolated complexes containing FGF2 and IgG, or fragments thereof, optionally where the complexes further contain VEGF, HGF, or fragments thereof, are administered in an amount required to achieve a therapeutic or prophylactic effect. Such an amount will vary depending on the conditions. Typically, biologically active isolated complexes will be purified and subsequently concentrated so that the protein content of the composition is increased by at least about 5-fold. 10-fold or 20-fold over the amount of protein originally present in the media. In other embodiments, the protein content is increased by at least about 25-fold. 30-fold. 40-fold or even by 50-fold. Preferably, the composition comprises an effective amount of isolated complexes containing FGF2 and IgG, or fragments thereof, optionally where the complexes further contain VEGF, HGF, or fragments thereof.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit vasoprotection, carioprotection, reduce vascular injury, or induce another desirable biological response. Such determinations do not require undue experimentation, but are routine and can be ascertained without undue experimentation

### Methods of Delivery

Compositions comprising isolated complexes containing FGF2 and IgG, or fragments thereof, optionally where the complexes further contain VEGF, HGF, or fragments thereof, may be delivered to a subject in need thereof. The compositions may be delivered as part of a standard of care procedure. Modes of administration include intramuscular, intra-cardiac, oral, rectal, topical, intraocular, buccal, intravaginal, intracisternal, intra-arterial, intracerebroventricular, intratracheal, nasal, transdermal, within/on implants. e.g., fibers such as collagen, osmotic pumps, or parenteral routes. The term “parenteral” includes subcutaneous, intravenous, intramuscular, intraperitoneal, intragonadal or infusion. In instances, administration of a complexes of the invention is associated with a long-term increase in cardiac perfusion.

The compositions can be administered via localized injection, including catheter administration systemic injection, localized injection, intravenous injection, or parenteral administration. When administering a therapeutic composition of the present invention, it will generally be formulated in a unit dosage injectable form (solution, suspension, emulsion). Dosages can be readily adjusted by those skilled in the art (e.g., a decrease in purity may require an increase in dosage). Compositions of the invention can be introduced by injection, catheter, or the like. Compositions of the invention include pharmaceutical compositions comprising cellular factors of the invention and a pharmaceutically acceptable carrier. Administration can be autologous or heterologous.

In embodiments, the compositions are infused from an indwelling percutaneous coronary intervention (PCI) guide catheter. The compositions can be infused before, after, or during stenting and/or restoration of blood flow. In instances, agents of the present invention (e.g., complexes containing FGF2 and IgG, or fragments thereof, optionally where the complexes further contain VEGF, HGF, or fragments thereof) are administered by intracoronary infusion, optionally from a percutaneous coronary intervention (PCI) guide catheter. It can be advantageous to administer the compositions of the invention to a coronary artery, optionally following myocardial infarction (MI).

### Methods for Evaluating Therapeutic Efficacy

In one approach, the efficacy of a treatment is evaluated by measuring, as a non-limiting example, vascular integrity. Such methods are standard in the art and are described herein (see. e.g., the Examples provided below). In particular, a method of the present invention, decreases vascular permeability by at least about 5%, 10%, 20%, 40%, 50%, 60%, 70%, 80%, 90%, 100%. In one embodiment, therapeutic efficacy is assessed by measuring a reduction in apoptosis. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example. Apo-ONE® Homogeneous Caspase-3/7 Assay. FragEL TUNEL kit (ONCOGENE RESEARCH PRODUCTS. San Diego, CA), the ApoBrdU DNA Fragmentation Assay (BIOVISION, Mountain View, CA), and the Quick Apoptotic DNA Ladder Detection Kit (BIOVISION, Mountain View, CA). In another embodiment, therapeutic efficacy is assessed by measuring cell proliferation (e.g., using a CyQUANT assay). In some instances, efficacy is measured using electrophysiological recordings (e.g., using a CoreMap high density electrode array or an electrocardiogram).

### Kits

The invention provides kits for the treatment or prevention of a condition associated with reperfusion injury, hypofusion, ischemic injury, and/or low/no-reflow (e.g. a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like). In one embodiment, the kit includes a therapeutic or prophylactic composition containing an effective amount of medium (e.g. concentrated human epicardial derived cell-conditioned medium) that contains complexes containing FGF2 and IgG, or fragments thereof, optionally where the complexes further contain VEGF, HGF, or fragments thereof, in unit dosage form. In some embodiments, the kit comprises a sterile container which contains a therapeutic or prophylactic composition of medium: such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, medium of the invention is provided together with instructions for administering the medium to a subject having or at risk of developing reperfusion injury, hypofusion, ischemic injury, and/or low/no-reflow or in need of reperfusion after myocardial ischemia. The instructions will generally include information about the use of the composition for the treatment or prevention of a condition associated with reperfusion injury, hypofusion, ischemic injury, and/or low/no-reflow (e.g., a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound, and the like). In other embodiments, the instructions include at least one of the following: description of the medium: dosage schedule and administration for treatment or prevention of a condition associated with reperfusion injury, hypofusion, ischemic injury, and/or low/no-reflow (e.g., a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound, and the like) or symptoms thereof: precautions; warnings: indications: counter-indications: over dosage information: adverse reactions: animal pharmacology: clinical studies: and/or references, the treatment regime, reagents, equipment (test tubes, reaction vessels, needles, syringes, etc.) and standards for calibrating or conducting the treatment. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook. 1989): “Oligonucleotide Synthesis” (Gait. 1984); “Animal Cell Culture” (Freshney. 1987): “Methods in Enzymology” “Handbook of Experimental Immunology” (Weir. 1996): “Gene Transfer Vectors for Mammalian Cells” (Miller and Calos. 1987): “Current Protocols in Molecular Biology” (Ausubel. 1987): “PCR: The Polymerase Chain Reaction”. (Mullis. 1994): “Current Protocols in Immunology” (Coligan. 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Large Animal (Pig) Model of MI with Reperfusion

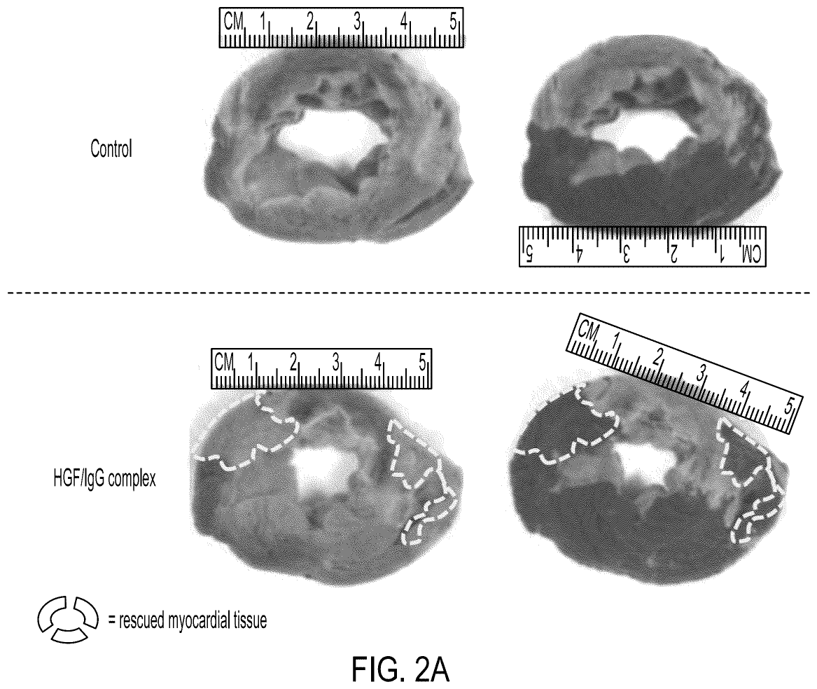

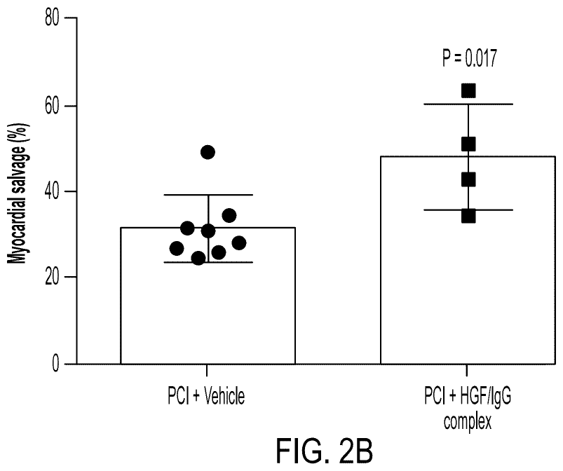

To evaluate the efficacy of an HGF/IgG complex in an animal model, the vaso- and cardioprotective effects of the compound were evaluated in a large animal model of myocardial infarction (MI) using catheters, balloons, and stents. Under anesthesia and with fluoroscopic guidance, a percutaneous coronary intervention (PCI) balloon catheter was advanced and inflated to completely occlude the left anterior descending coronary artery (LAD) for 60 min prior to revascularization. At the time of reperfusion (balloon deflation/stenting), pigs in the control group received contrast dye and 12.5 mls of vehicle (DMEM/F12 base medium) from the percutaneous coronary intervention (PCI) guide catheter, just prior to its removal. Pigs in the HGF/IgG complex treatment group received contrast and 12.5 mls of DMEM/F12 containing HGF/IgG complexes (63 μg of active human HGF with 100 μg of pig IgG). After 24 hrs. all pigs were infused with Evan's blue dye to determine Area At Risk (AAR) and euthanized. Hearts were removed and transversely cut from apex to base (1 cm slices), then stained with (2.3.5-triphenyltetrazolium chloride, a.k.a. TTC), and digitally photographed (FIGS. 2A and 2B). Areas of viable and scarred cardiac tissue were quantified by a blinded observer with software (Scion Image 4.0.2). Pigs that underwent PCI and received HGF/IgG complexes had significantly more myocardial salvage than did pigs that received PCI and vehicle (48±12% rescue of AAR. N=4-8, P=0.017; FIGS. 2A and 2B). Studies were also performed with HGF/IgG complex doses containing 32.5 μg HGF (23±6% rescue of AAR. N=6 pigs) and 125 μg HGF (36±12% rescue of AAR. N=11 pigs); but the 63 μg intracoronary dose was the most effective. This is the dose of used in Example 7 below.

### Example 2: Multiple HGF/IgG Complex Treatments Did not Elicit Production of Auto-HGF Antibodies

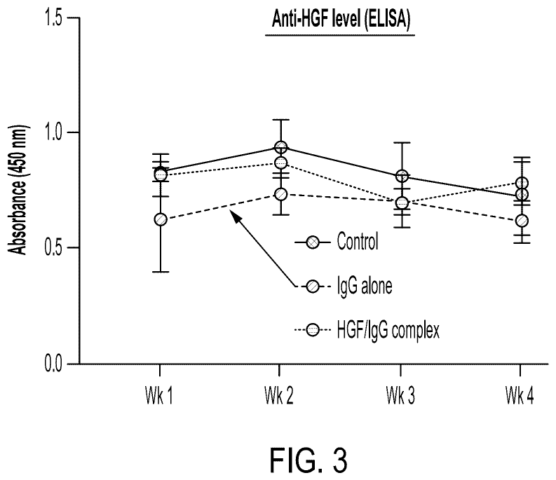

Immunogenicity is an important issue for use of antibody-based therapeutics in patients. Thus, in the context of myocardial infarction (MI) with reperfusion, it was determined whether multiple administrations of HGF/IgG would lead to production of auto-antibodies against HGF. Adult rats underwent myocardial infarction (MI) surgery and received intracardiac infusion of HGF/IgG complexes (10 μg, rat HGF in complex with 16 μg rat IgG) at reperfusion. In addition, all rats received IV (tail vein) booster injections of HGF/IgG complexes on days 3 and 5 after MI. Blood plasma was collected weekly and tested for anti-rat HGF by ELISA. Despite multiple doses. HGF/IgG complexes did not provoke an immune response (FIG. 3).

### Example 3: FGF2 Seeded Formation of FGF2:IgG, FGF2:HGF:IgG, and FGF2:VEGF:IgG Complexes without Ultra Centrifugal Units/Centrifugation

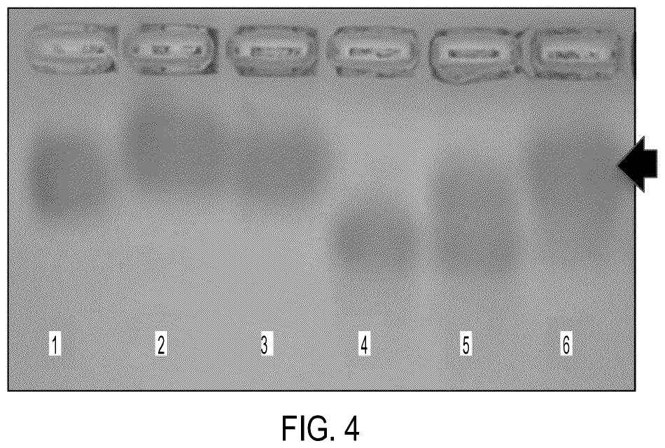

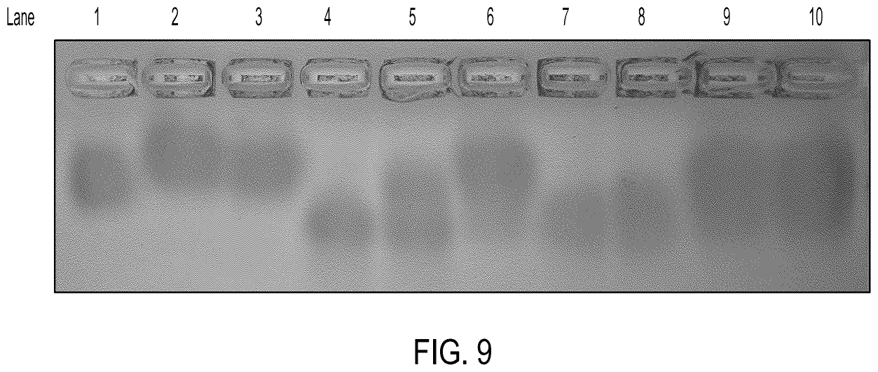

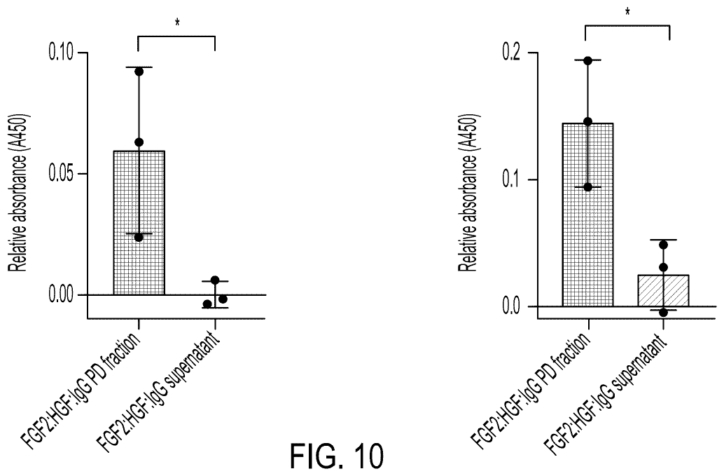

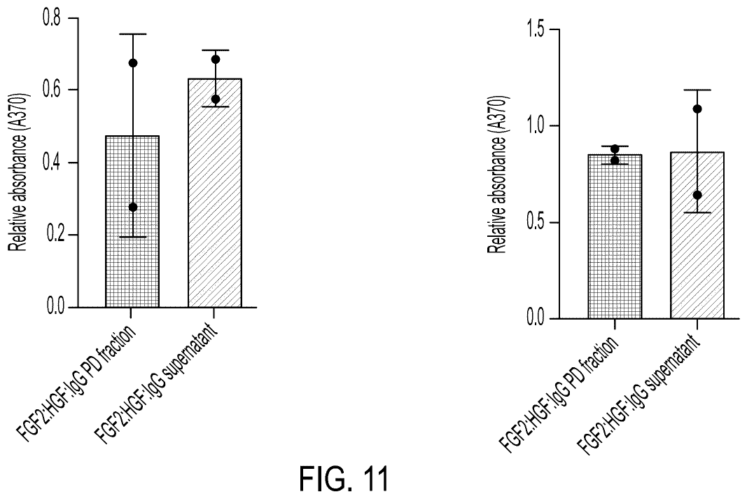

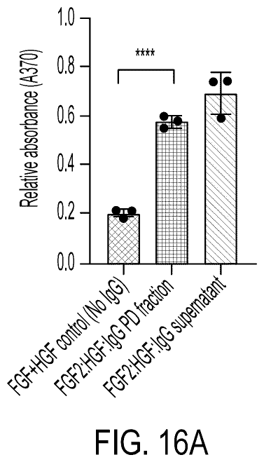

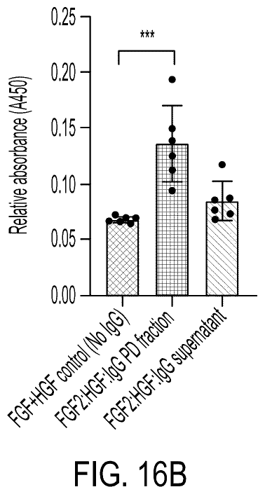

To identify complexes with improved properties, numerous different growth factors and cytokines were evaluated for their ability to interact with IgG. Only FGF2 was found to interact with IgG. Preparation of the HGF/IgG complex required centrifugation in Amicon units to a 50-fold concentration, whereas preparation of a FGF2:IgG complex formed spontaneously without any need for any centrifugation/concentration step. Addition of FGF2 to HGF and IgG in a 1:1:2 molar ratio promoted spontaneous formation of FGF2:HGF:IgG (FIGS. 4 and 9). Complexes so formed were denoted using colons (:), as in FGF2:HGF:IgG, to indicate that the complexes formed spontaneously (i.e., without any concentration/centrifugation step). The ability of FGF2 to seed complex formation with growth factors other than HGF was also evaluated, and it was found that FGF2 can also seed FGF2:VEGF:IgG complex formation (FIG. 9), where VEGF is vascular endothelial growth factor. FGF2:HGF:IgG complex formation was confirmed using pulldown assays (FIGS. 10, 11, 16A and 16B).

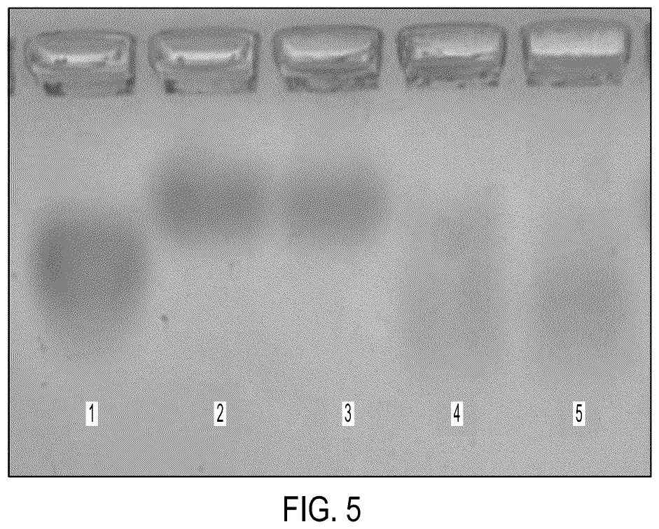

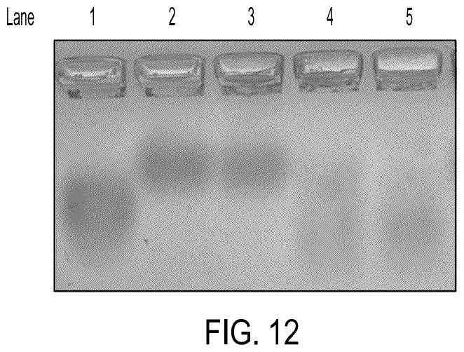

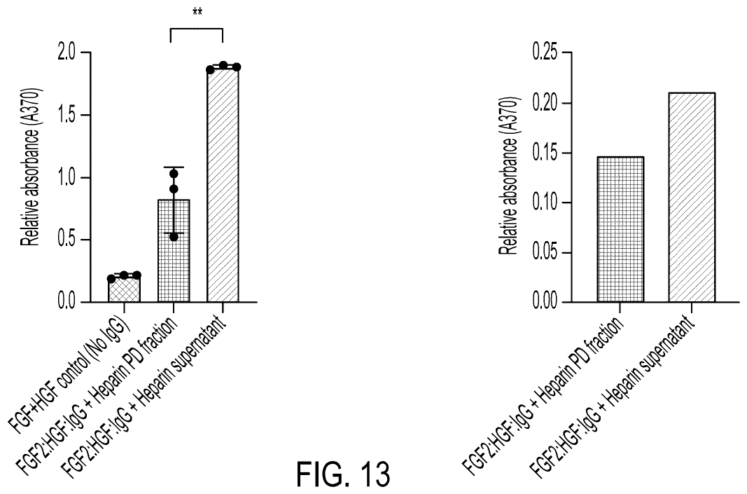

Stoichiometric addition of heparin prevented complexation and disassociates pre-existing complexes for FGF2:IgG (FIGS. 5 and 12). HGF/IgG, and also FGF2:HGF:IgG. These data suggested that FGF2 and HGF interacted with the IgG Fc domain via their heparin binding peptide sequences. Co-incubation with heparin liberated growth factors from FGF2:HGF:IgG complexes FIG. 13).

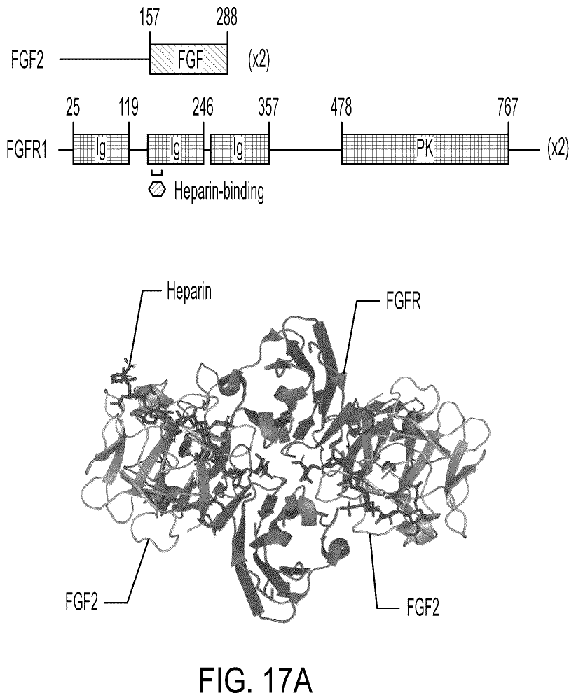

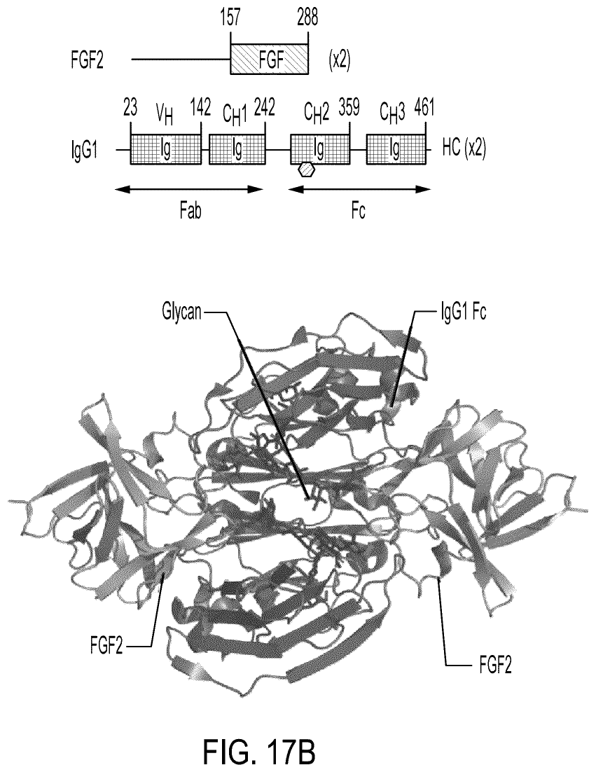

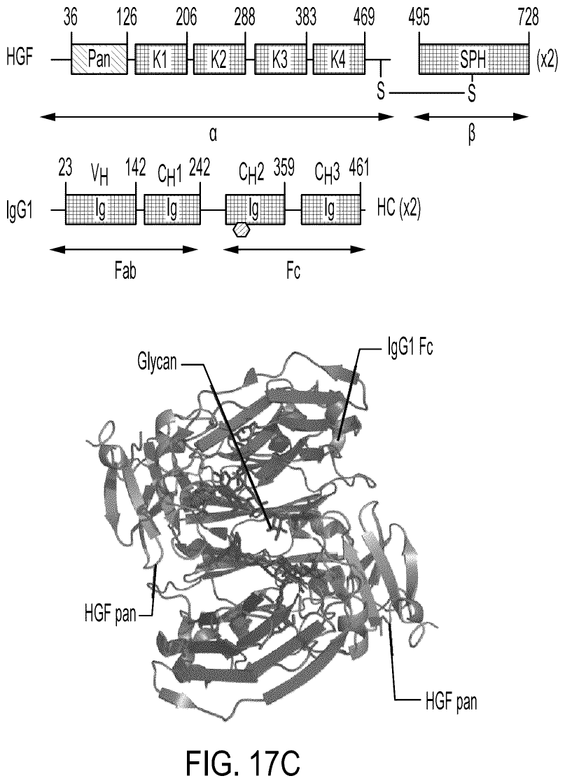

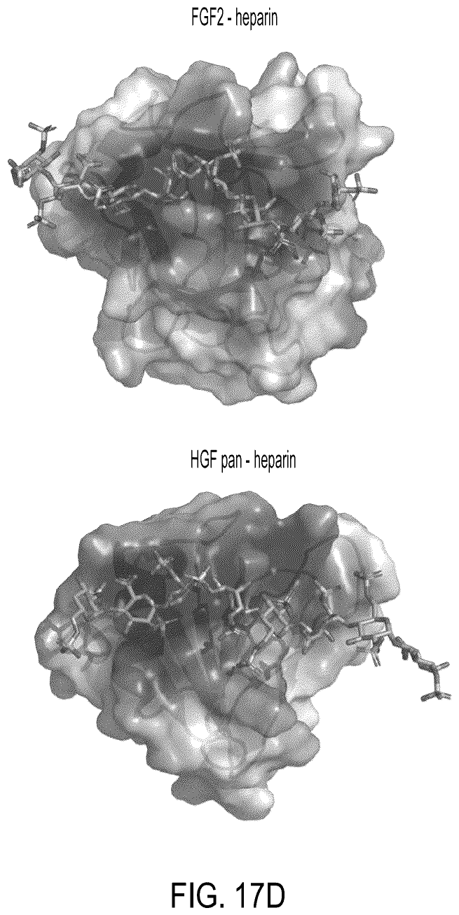

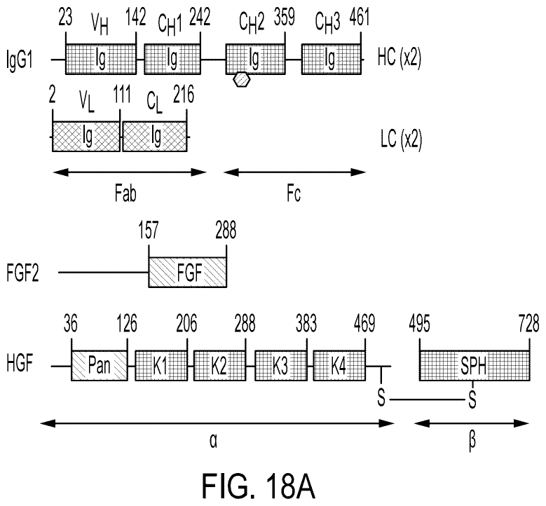

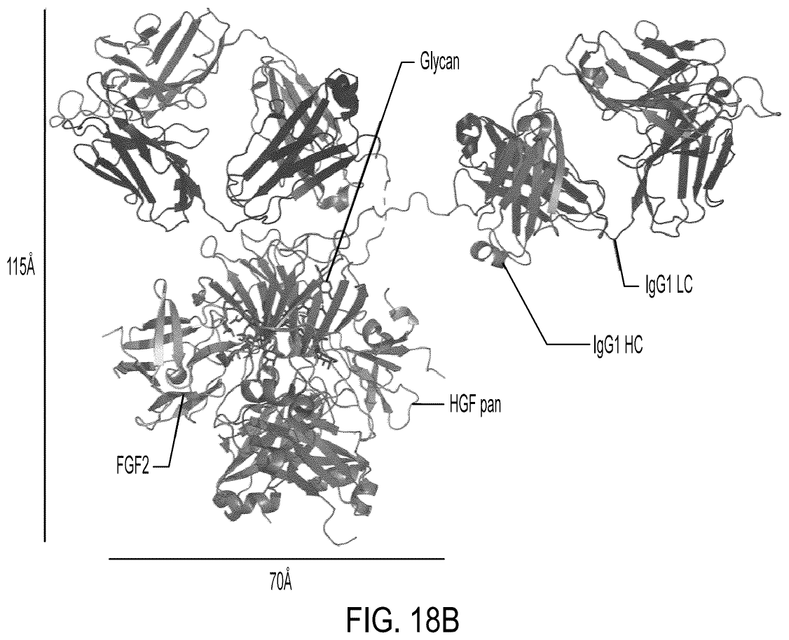

Three-dimensional models were prepared to further investigate the complexes (FIGS. 17A-17D and 18A-18B).

### Example 4: HGF/IgG and FGF2:HGF:IgG Complexes Both Protected Human Microvascular Endothelial Cells Under Conditions that Mimicked Tissue Ischemia

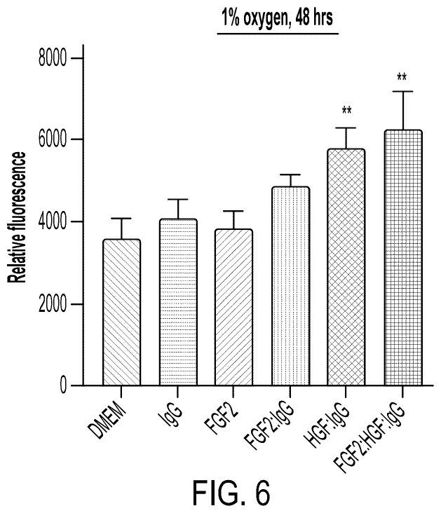

To compare efficacy of HGF/IgG and FGF2:HGF:IgG complexes, cell protection assays were performed with primary human cardiac microvascular endothelial cells under culture conditions that simulated ischemia (nutrient deprivation and 1% oxygen). By Cyquant assay, after 48 hrs of simulated ischemia, significant protection conferred by HGF/IgG and FGF:HGF:IgG complexes were detected, as compared with DMEM/F12 base medium (vehicle). IgG alone. FGF2 alone, or FGF2:IgG complex (DMEM vs. HGF/IgG. P<0.01; DMEM vs. FGF2:HGF:IgG. P<0.001; FIG. 6).

### Example 5: Biochemical Pulldown Assays Showed FGF:HGF:IgG Complexes were Stable for Over Time

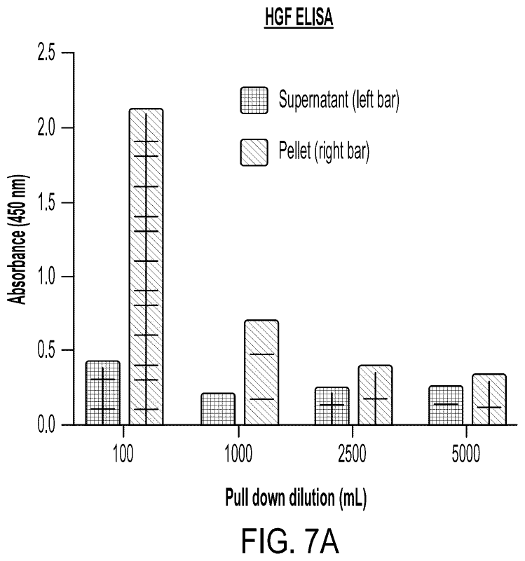

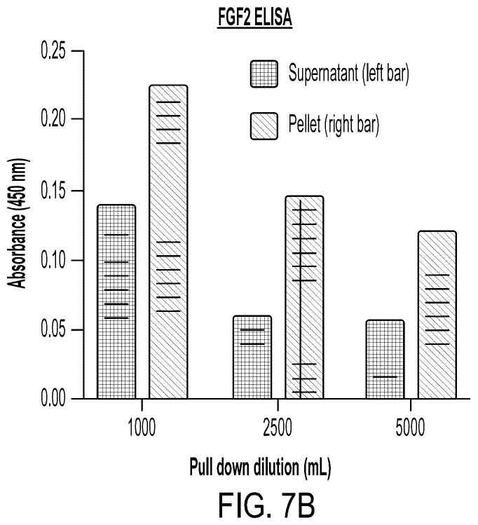

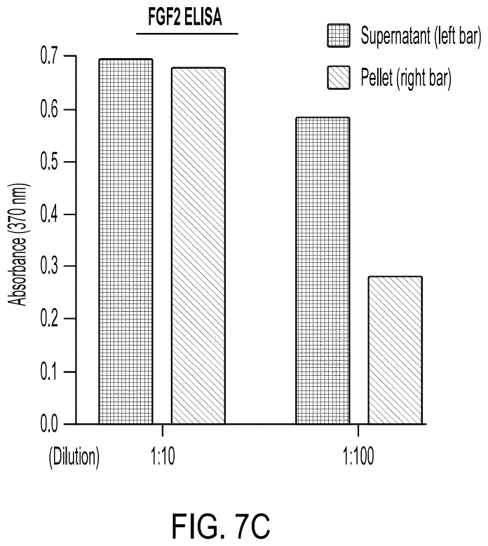

To evaluate stability of the FGF2:HGF:IgG complexes, basic fibroblast growth factor (FGF2), hepatocyte growth factor (HGF), and IgG were incubated in a 1:1:2 molar ratio for 2 hrs at room temperature. The resulting complexes were then stored at 4-6° ° C. for 2 days. Then, pulldowns were performed using Protein A-Sepharose beads, which bind the Fc region of IgG1 (FIGS. 7A and 7B). After incubation, centrifugation, and phosphate buffered saline (PBS) washes, sodium deoxycholate (1%) detergent was used to liberate bound factors from the beads. Commercial enzyme-linked immunosorbent assays (ELISAs) for FGF2 and HGF (R & D Systems) were then used to measure unbound factors (present in the supernatants) and also factors in complexes (present in pellets). Pulldowns were also done using a biotinylated antibody directed against a 6×His-tag (SEQ ID NO: 12) located in the N-terminus of a human HGF used in the experiment (FIG. 7C). In this case, detection of FGF2 (which lacked a His-tag) confirmed that HGF and FGF2 were present in the same complexes (FIG. 7C). The FGF:HGF:IgG complexes were stable for at least 2 days at 4-6° C. These quantitative Pulldown assays provide a method to determine binding kinetics for FGF2/IgG and/or FGF2:HGF:IgG complexes in Example 8 below.

### Example 6: Treatment with FGF2:HGF:IgG Complex Preserved Functional Myocardial Tissue within an Area of Risk after MI

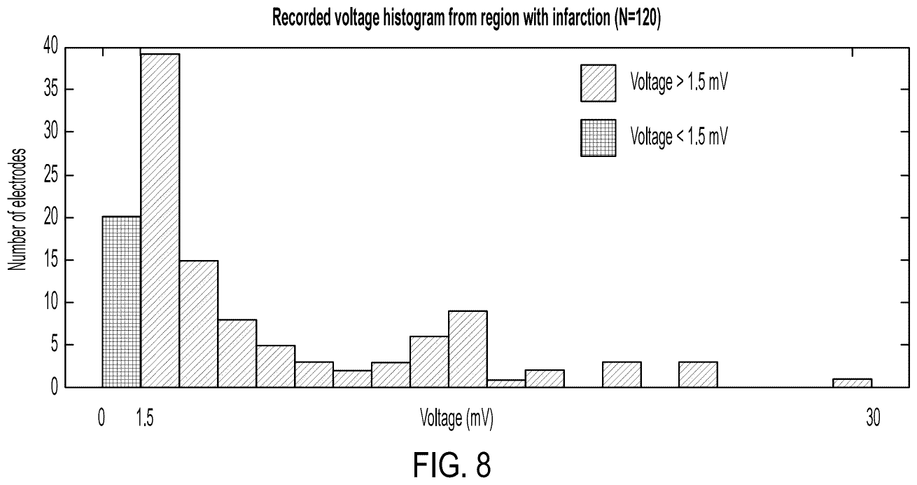

Using CoreMap's sensitive electrode array system, it was demonstrated that myocardial tissue saved by treatment with the FGF2:HGF:IgG complex at 24 hrs after myocardial infarction (MI) and reperfusion was electrically active, and not arrhythmic (FIG. 8). The complex was delivered from a percutaneous coronary intervention (PCI) guide catheter after a myocardial infarction (MI). For epicardial measures, individual electrode recordings with voltages above 1.5 mV were considered to be functionally “normal”. Remarkably, in regions with infarction, 83% of electrodes recorded voltages at or above 1.5 mV in a FGF2:HGF:IgG complex-treated heart (N=120 individual electrodes. FIG. 8).

### Example 7: Long-Term Effects of Intracoronary Treatment with HGF/IgG or FGF2:HGF:IgG Complexes on Cardiac Structure and Function after MI and PCI

Pre-clinical large animal studies are preformed to compare FGF2:HGF:IgG complexes to HGF/IgG complexes. The studies allow for determination as to whether FGF2:HGF:IgG and/or HGF/IgG has advantages in terms of efficacy and commercialization potential. The studies shed light on the long-term effects of intracoronary treatment with the complexes on cardiac structure and function after MI. The studies compare FGF2:HGF:IgG and/or HGF/IgG treated pigs with control (vehicle-treated) pigs in regard to cardiac tissue survival, angiogenesis, remodeling, and function after MI.

A balloon catheter is inserted retrograde from a femoral artery in order to occlude the left anterior descending coronary artery (LAD) just distal to its first diagonal branch for 60 min in commercial farm swine (females. 50 kg: N=30). Immediately after reperfusion, all treatment and control infusions are delivered slowly (over 2 min) from the guide catheter into the LAD. Group 1 (N=10. Control) receives vehicle (DMEM/F12). Group 2 (N=10) receives HGF/IgG complexes. Group 3 (N=10) receives FGF2:HGF:IgG complexes. To determine cardiac function, all pigs undergo two-dimensional echocardiography (echo) at baseline (prior to LAD occlusion), at 1 week, and at 1 month after MI. All pigs have blood samples taken 1 hr prior to occlusion. 1 hr after occlusion, at 24, 48, and 72 hrs after occlusion, and weekly thereafter, for ELISAs to quantify cTnI and Angpt-2 levels as measures of infarct size and vascular endothelial injury, respectively. All pigs are euthanized 1 month after MI and the last echo. The pig hearts are serially sliced (5 mm) and alternating slices are reserved for proteomics. The other slices are fixed in 10% formalin and paraffin-processed for histology, analysis of fibrosis, and immunohistochemistry (CD31, smooth muscle actin) to quantify blood vessels. Steriology is used for unbiased quantification (Microbrightfield StereoInvestigator).

As pilot study, 2 pigs were treated with the FGF2:HGF:IgG complex. The administration protected as well as the HGF/IgG complex.

Not being limited by theory, the FGF2:HGF:IgG complex provides greater benefit at the 1 month time point than HGF/IgG due to improved angiogenesis and perfusion.

Treatments are deemed successful if they reduce final infarct size by 25% or more at 1 month after MI, significantly reduce cardiac fibrosis and negative remodeling, and significantly improve myocardial perfusion and cardiac functional parameters as measured by echocardiography (e.g. ejection fraction, cardiac output, wall motion). Data gathered demonstrates significant long-term benefit to cardiac structure and function conferred by HGF/IgG- and/or FGF2:HGF:IgG-treatment in a pre-clinical, large animal model of MI with reperfusion. This data provides support for treatment studies with a 3 month endpoint and evaluations using high resolution Cardiac Magnetic Resonance (CMR) imaging.

### Example 8: Optimizing Conditions to Form and Maintain Complexes

In the above examples, a 1:1:2 stoichiometry was used to prepare FGF2:HGF:IgG complexes. However, the specific quantity of factors or IgG that were free or within complexes under different conditions was not evaluated. The kinetics of dissociation/association of components of the complexes (e.g., HGF/IgG and FGF2:HGF:IgG) is determined under defined conditions of temperature, osmolarity, and pH to inform the optimization of formulations, generate the complexes more efficiently, and provide compositions with a good safety profile.

Relative concentrations of binding partners are varied and use the Pulldown/ELISA system described in Example 5 above is to determine dissociation equilibrium constants (i.e. KD=Koff/Kon) for FGF2:IgG. HGF/IgG, and FGF2:HGF:IgG. To better estimate affinity, binding studies are preformed using a matrix of incubation times and concentrations. Assays are also performed with fluorescently-labeled factors that are displaced by adding non-labeled factors or heparin. Following/during displacement, measurements are taken of what remains in the supernatant after Pulldowns.

Optimal conditions for producing the HGF/IgG complex and for producing the FGF2:HGF:IgG complex are determined in terms of input ratio for components, total concentrations, and incubation times required to facilitate complexation. The conditions increase efficiency for preparation of stable complexes. Optimal conditions are also determined for storing the complexes. Conditions evaluated include, among others, amount of reagent used, incubation/storage times, and temperature (e.g., refrigeration).

Further, conditions resulting in aggregation of the protein complexes are determined. For safety, it is important to know if and when aggregation may occur. To evaluate conditions resulting in aggregation, experiments are conducted with high molar concentrations and complexes are characterized using a Twin laser ZetaView Particle Analyzer. The ZetaView Particle Analyzer provides quantitative data for particle size distribution.

Next, the effects of Fc domain glycosylation on complex formation are determined. Heparin interferes with complex formation and dissociates pre-existing complexes. Determining the role of IgG glycosylation is important because a particular Fc glycoform may provide insights into optimal means to generate complexes (e.g., HGF/IgG and FGF2:HGF:IgG).

Non-specific, polyclonal Human IgG1 (Sigma) is treated with enzymes (i.e. glycosidases) to remove sugar moieties from the Fc domain. Commercial enzyme kits are used (deGlycIT and SialEXO kits. Genovis, Cambridge, MA). Both kits conveniently provide active enzymes covalently-conjugated to agarose beads. After incubation and centrifugation. Fc glycan-depleted IgGs are collected from the supernatant and used to form FGF2:IgG, HGF/IgG, or FGF2:HGF:IgG complexes. Biochemical pulldowns and enzyme-linked immunosorbent assays (ELISAs) are used to evaluate whether removal of N-glycans, O-glycans, or sialoglycans affects complexation of IgG with FGF2 and/or HGF.

Complete removal of sugars from IgG1 abolishes complexation, whereas desialation reduced complexation but not block complexation entirely.

### Example 9: Efficacy of FGF2:HGF:IgG Complexes in an Animal Model

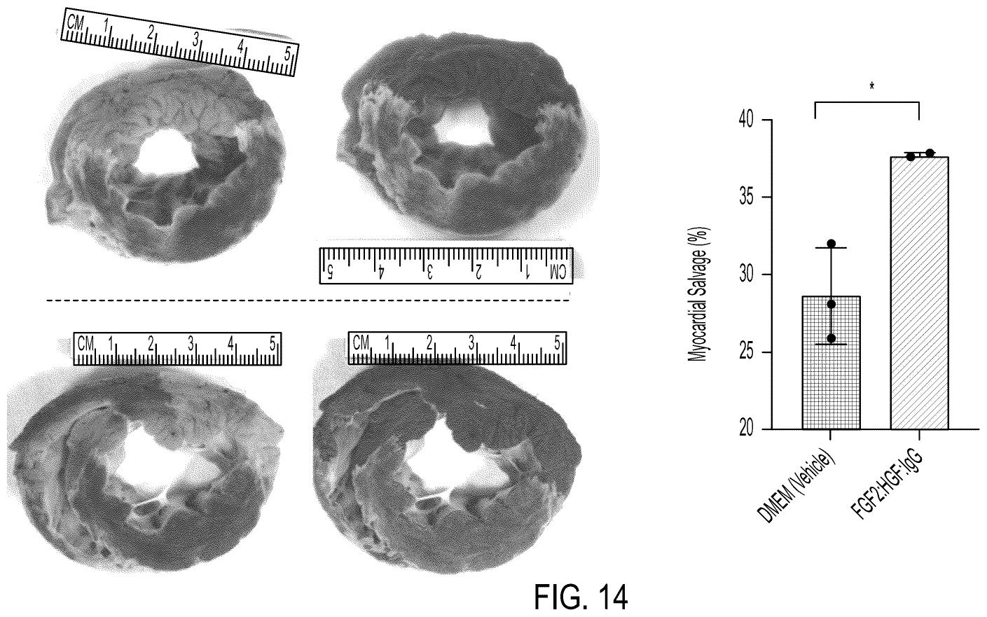

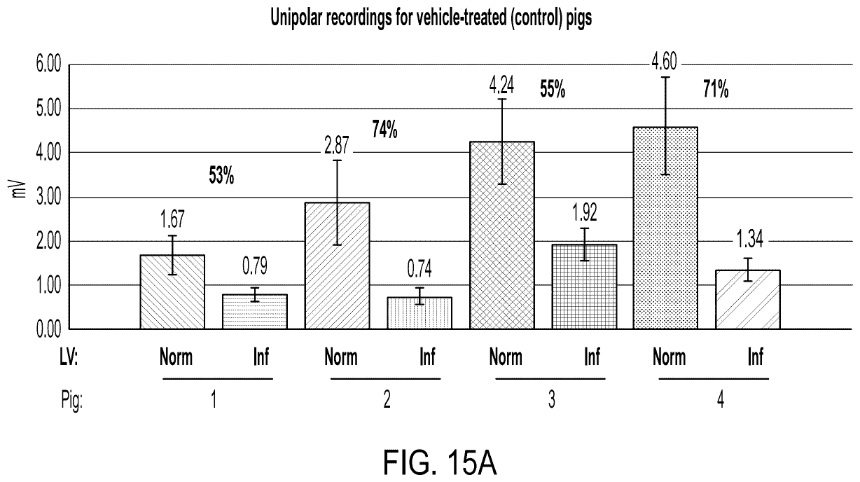

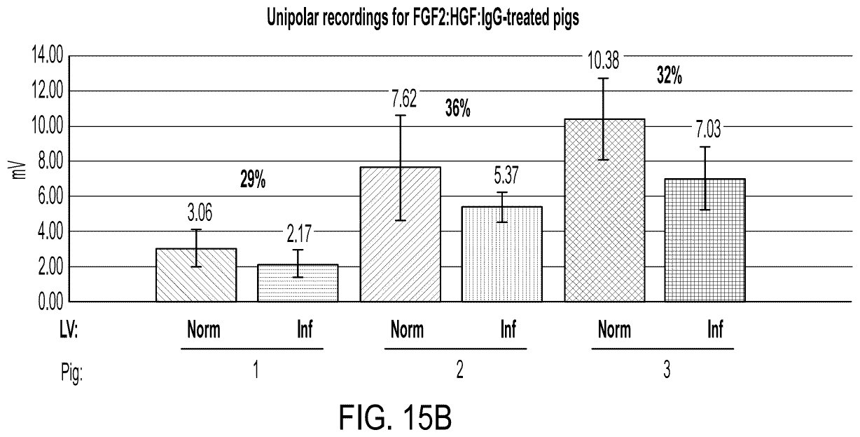

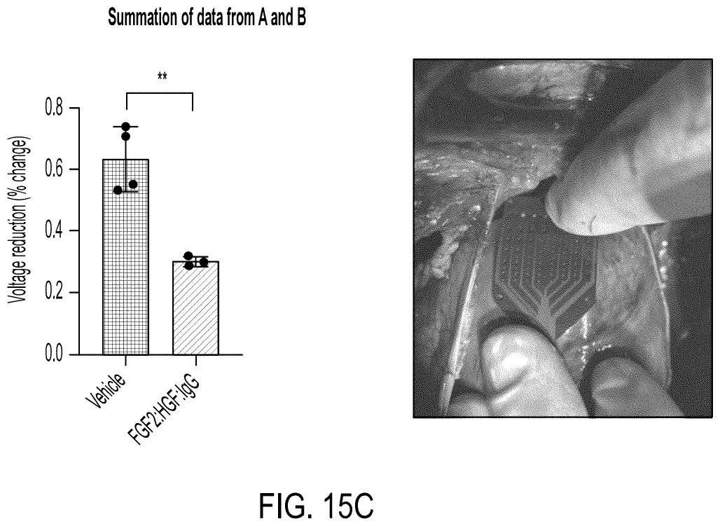

The efficacy of the FGF2:HGF:IgG complex was evaluated and demonstrated in pigs having suffered a myocardial infarction (MI). Intracoronary delivery of FGF2:HGF:IgG complexes preserved jeopardized myocardium after myocardial infarction (MI) with reperfusion (FIG. 14). Efficacy was further confirmed using electrophysiological recordings using the CoreMap high density electrode array (FIGS. 15A-15C).

### Example 10: FGF2/HGF/IgG and FGF2:HGF:IgG Complexes Protected Human Cardiac Microvascular Endothelial Cells Against Simulated Ischemia

The efficacy of FGF2/HGF/IgG and FGF2:HGF:IgG in protecting human cardiac microvascular endothelial cells against simulated ischemia was evaluated. Regarding the notation used to specify the complexes throughout the present disclosure, backslashes (/) indicate a complex formed by a method involving concentrating the components of the complex (e.g., by an ultracentrifugation method), and colons (:) indicate a complex formed by a method involving no such concentrating step (e.g., the complex forms spontaneously without any need for concentrating the reagents).

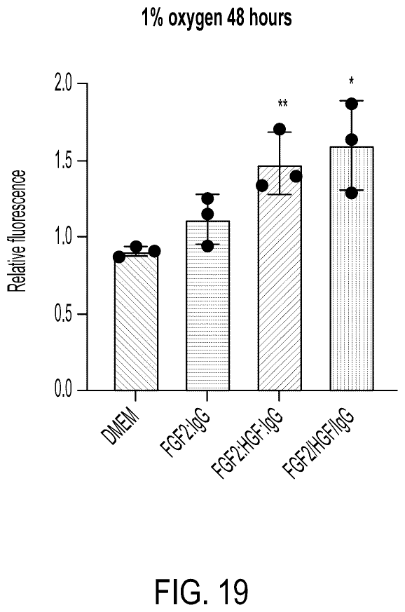

Using the commercially available Cy Quant assay, it was determined that FGF/HGF/IgG (Amicon-concentrated) complexes and FGF2:HGF:IgG (spontaneous formation) complexes protect human cardiac microvascular endothelial cells under conditions simulating ischemia (1% oxygen and nutrient deprivation) (FIG. 19).

### METHODS OF THE EXAMPLES

The following methods were employed in the above examples.

### HGF/IgG Complex Formation

In Examples 1-6, The HGF/IgG complex was prepared by mixing IgG with HGF at a molar ratio of 1:1, followed by concentrating the mixture by about at least 40-fold using centrifugation and Amicon units. The backslash (/) in the notation HGF/IgG was used to indicate that the complex was formed using an centrifugation/concentration step.

The following statistic methods are employed in Examples 7 and 8.

### Data Analysis

All data from experimental treatments and controls are tested statistically (i.e. by linear or non-linear regression, ANOVA with post-hoc tests, or Student's T-test). For ANOVA, Bonferroni correlation is used in most cases. For data that are not normally-distributed the Wilcoxon rank sum test (MannWhitney U) or Kruskal-Wallis ANOVA and the Dunn procedure are used.

### Statistical Power

Six to eight pigs are adequate to detect differences for treatments with HGF/IgG and/or FGF2:HGF:IgG compared with vehicle-treated controls. Based on estimated losses of 10% during MI procedure (e.g. arrhythmia during ischemic period), plus an additional 10% during the month following myocardial infarction (MI), N=10 is used. For assay of plasma Angpt-2 levels, N=13 enabled detection of a highly significant correlation with infarct size in ST segment elevation myocardial infarction (STEMI) patients at 48 hr post event (P<0.002, FIG. 1). Thus, 48 hr plasma Angpt-2 and cardiac Troponin I (cTnI) ELISA data from N=10 pigs is sufficient. N=10 pigs allows for determination of treatment effects on electrical activity (using CoreMap's sensitive electrode array) as well as myocardial perfusion and cardiac function (by echocardiography).

### Sex as a Biological Variable

Due to territorialism and aggression in male swine (and the need to castrate males for long-term housing), only adult female pigs (˜50 kg) are used.

### Experimental Rigor and Reproducibility

The surgeon is blinded to treatment identity, as are individuals involved with echocardiographic and electrophysiologic evaluations. Similarly, for assays such as immunohistochemistry, all observers are blinded to slide (sample) identity to allow for objective cell or vessel counts, etc. In Example 8, all assays are performed in triplicate for accuracy and replicated a minimum of 3 or more times.

### Procedure for Myocardial Infarction (MI) in Swine

Details of anesthesia, intra-coronary access and intra-coronary balloon inflation are provided in Meyer et al. (JACC: Cardiovascular Interventions 2009:2:216-221). Briefly, female swine (˜50 kg) are used. The animals are pre-medicated with ketamine (20 mg/kg s.c.) and anesthetized with isoflurane. After induction of anesthesia, they undergo endotracheal intubation and are ventilated with a large animal ventilator (100% O₂). A limb lead electrocardiogram (ECG) is monitored throughout the procedure. Using digital flurosocopy, an angioplasty catheter is advanced into the left anterior descending coronary artery (LAD) via a femoral sheath and its balloon is inflated just proximal to the second diagonal branch for 60 minutes. The balloon is then deflated, and Dulbecco's modified eagle medium (DMEM: vehicle control) or protein complexes (e.g., FGF2:HGF:IgG and/or HGF/IgG) in vehicle are infused into the LAD. The catheter is removed and an arterial closure device is deployed. The animal is monitored continuously following coronary occlusion until anesthesia has worn off and then transferred to a housing facility. Ventricular fibrillation occurs infrequently and is treated promptly with DC cardioversion if it occurs during active monitoring. The 10 week post-MI survival rate is about 80% with LAD occlusions placed after the second diagonal LAD branch, with 10% of the losses during the MI procedure (prior to treatment), and 10% losses during the month following MI.

### Authentication

The specificity of antibodies is validated by Western blotting with molecular weight standards and ladders to confirm that bands of the expected size are identified and quantified. For SDS-PAGE gels, equal loading is based on either A) Protein determination or B) Equal cell number, or C) Equal wet weight of tissue. Different Western Blots are performed, in many instances, using multiple primary antibodies against a given protein or peptide antigen of interest. This further ensures specificity. Protein bands detected on film are quantified by densitometry using a digital scanner and ImageJ. Protein data are normalized against those for housekeeping proteins such as beta actin (gels) or myh6 (proteomics). For immunohistochemistry, isotype control stains are performed for each isotype and host species that is used.

The following methods were used in Example 7.

### HGF/IgG Complex Preparation

The HGF/IgG complex is prepared by combining recombinant human HGF in a 1:1 molar ratio with porcine IgG in DMEM/F12, sterile-filtered, and then concentrated 50-fold using Amicon devices (Rao K S, Aronshtam A, McElroy-Yaggy K L, Bakondi B, VanBuren P, Sobel B E, Spees J L. Human epicardial cell-conditioned medium contains HGF/IgG complexes that phosphory late RYK and protect against vascular injury. Cardiovasc Res. 107:277-286 (2015)). The HGF/IgG complex is diluted in 12.5 ml of DMEM/F12 to an HGF dose of 0.063 mg/50 kg pig prior to administration.

### FGF2:HGF:IgG Complex Preparation

The FGF2:HGF:IgG complex is prepared by combining recombinant human FGF2 and HGF with porcine IgG at a 1:1:2 molar ratio in 150 microliters of DMEM/F12. The FGF2:HGF:IgG complex delivers a combined dose of FGF2 (0.0125 mg/kg), HGF (0.063 mg/50 kg), and IgG (0.209 mg/kg) in DMEM/F12 (12.5 ml).

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The invention may be related to Rao, K., et al. “Human epicardial cell-conditioned medium contains HGF/IgG complexes that phosphorylate RYK and protect against vascular injury”, Cardiovascular Res., 107:277-286 (2015); to Rao, Krithika, “Epicardial Cell Engraftment And Signaling Promote Cardiac Repair After Myocardial Infarction” (2016). Graduate College Dissertations and Theses. 479; and to U.S. Pat. No. 10,239,926 B2, the entirety of which are incorporated herein by reference for all purposes.

What is claimed is:

1. An isolated complex comprising a basic fibroblast growth factor (FGF2) polypeptide, or a fragment thereof, and an immunoglobulin G (IgG) polypeptide, or a fragment thereof.

2. The isolated complex of claim 1 further comprising an additional growth factor polypeptide, or a fragment thereof.

3. The isolated complex of claim 2, wherein the additional growth factor comprises a hepatocyte growth factor (HGF) polypeptide, VEGF, or a fragment thereof.

4. The isolated complex of claim 1, wherein the polypeptides are complexed by non-covalent interactions.

5. The isolated complex of claim 1, wherein the complex does not comprise an antibody-antigen interaction.

6. A composition comprising the complex of claim 1.

7. A pharmaceutical composition for increasing vascular integrity, promoting angiogenesis, and/or preserving cardiac tissue, the composition comprising the complex of claim 1 and a pharmaceutically acceptable excipient.

8. A method for producing a complex, the method comprising contacting an isolated fibroblast growth factor (FGF2) polypeptide or a fragment thereof with an immunoglobulin G (IgG) polypeptide, or a fragment thereof, thereby forming the complex, wherein the method does not comprise any concentrating step.

9. The method of claim 8, wherein the complex further comprises an additional growth factor polypeptide, or a fragment thereof.

10. The method of claim 9, wherein the additional growth factor comprises a hepatocyte growth factor (HGF), VEGF, or a fragment thereof.

11. The method of claim 8, wherein the complex does not comprise an antibody-antigen interaction.

12. The method of claim 8, wherein polypeptides of the complex are associated with one another by only non-covalent interactions.

13. A method for reducing cell damage or cell death following an ischemic event with reperfusion, the method comprising contacting a cell with the complex of claim 1, thereby reducing cell damage or cell death following the ischemic event with reperfusion.

14. The method of claim 13, wherein the ischemic event is associated with reperfusion injury, hypofusion, ischemic injury, and/or no/low-reflow.

15. The method of claim 13, wherein the ischemic event is associated with a myocardial infarction.

16. A method for increasing vascular integrity, promoting angiogenesis, and/or preserving tissue in a subject following an ischemic event with reperfusion, the method comprising administering to the subject the complex of claim 1, thereby increasing vascular integrity, promoting angiogenesis, and/or preserving cardiac tissue relative to a reference.

17. The method of claim 16, wherein the administration is associated with a reduction in vascular permeability relative to a reference.

18. A method for reducing vascular permeability in a subject following an ischemic event with reperfusion, the method comprising administering to the subject the complex of claim 1, thereby reducing vascular permeability relative to a reference.

19. The method of claim 16, wherein the administration is associated with an increases in vascular integrity or a reduction in death of cells.

20. The method of claim 16, wherein cells comprise an endothelial cell, microglial cell, blood-derived cell, smooth muscle cell, fibroblast, cardiac myocyte, skeletal muscle cell, peripheral neuron, CNS neuron, astrocyte, oligodendrocyte, pulmonary epithelial cell, liver epithelial cell, or kidney epithelial cell.
